SEQUENCE LISTING (36 sequences)

SEQ ID NO: 1 - DNA, 2710 bp, Homo sapiens
ccgccatggtccccgacaccgcctgcgttcttctgctcaccctggctgccctcggcgcgtccggacagggccagagcccgttgggtaagccgcgttagcacccgcgccgtgcccacggccccacaacggactgtaggacccgtgagaggcccgggatccaggctgtttggggctcacggactgttcgtaggggacgtgccgggcgcagaaagcaggtggcgggaccgagactagaggagcgcagtggggcctcggaggtccgggttcgctgcaacggtgggagttggtggtgggattccccggccccatgacgcctcaccaggtcccctgccgccgcaggctcagacctgggcccgcagatgcttcgggaactgcaggaaaccaacgcggcgctgcaggacgtgcgggagctgctgcggcagcaggtcagggagatcacgttcctgaaaaacacggtgatggagtgtgacgcgtgcgggatgcagcagtcagtacgcaccggcctacccagcgtgcggcccctgctccactgcgcgcccggcttctgcttccccggcgtggcctgcatccagacggagagcggcgcgcgctgcggcccctgccccgcgggcttcacgggcaacggctcgcactgcaccgacgtcaacgagtgcaacgcccacccctgcttcccccgagtccgctgtatcaacaccagcccggggttccgctgcgaggcttgcccgccggggtacagcggccccacccaccagggcgtggggctggctttcgccaaggccaacaagcaggtttgcacggacatcaacgagtgtgagaccgggcaacataactgcgtccccaactccgtgtgcatcaacacccggggctccttccagtgcggcccgtgccagcccggcttcgtgggcgaccaggcgtccggctgccagcggcgcgcacagcgcttctgccccgacggctcgcccagcgagtgccacgagcatgcagactgcgtcctagagcgcgatggctcgcggtcgtgcgtgtgtgccgttggctgggccggcaacgggatcctctgtggtcgcgacactgacctagacggcttcccggacgagaagctgcgctgcccggagcgccagtgccgtaaggacaactgcgtgactgtgcccaactcagggcaggaggatgtggaccgcgatggcatcggagacgcctgcgatccggatgccgacggggacggggtccccaatgaaaaggacaactgcccgctggtgcggaacccagaccagcgcaacacggacgaggacaagtggggcgatgcgtgcgacaactgccggtcccagaagaacgacgaccaaaaggacacagaccaggacggccggggcgatgcgtgcgacgacgacatcgacggcgaccggatccgcaaccaggccgacaactgccctagggtacccaactcagaccagaaggacagtgatggcgatggtataggggatgcctgtgacaactgtccccagaagagcaacccggatcaggcggatgtggaccacgactttgtgggagatgcttgtgacagcgatcaagaccaggatggagacggacatcaggactctcgggacaactgtcccacggtgcctaacagtgcccaggaggactcagaccacgatggccagggtgatgcctgcgacgacgacgacgacaatgacggagtccctgacagtcgggacaactgccgcctggtgcctaaccccggccaggaggacgcggacagggacggcgtgggcgacgtgtgccaggacgactttgatgcagacaaggtggtagacaagatcgacgtgtgtccggagaacgctgaagtcacgctcaccgacttcagggccttccagacagtcgtgctggacccggagggtgacgcgcagattgaccccaactgggtggtgctcaaccagggaagggagatcgtgcagacaatgaacagcgacccaggcctggctgtgggttacactgccttcaatggcgtggacttcgagggcacgttccatgtgaacacggtcacggatgacgactatgcgggcttcatctttggctaccaggacagctccagcttctacgtggtcatgtggaagcagatggagcaaacgtattggcaggcgaaccccttccgtgctgtggccgagcctggcatccaactcaaggctgtgaagtcttccacaggccccggggaacagctgcggaacgctctgtggcatacaggagacacagagtcccaggtgcggctgctgtggaaggacccgcgaaacgtgggttggaaggacaagaagtcctatcgttggttcctgcagcaccggccccaagtgggctacatcagggtgcgattctatgagggccctgagctggtggccgacagcaacgtggtcttggacacaaccatgcggggtggccgcctgggggtcttctgcttctcccaggagaacatcatctgggccaacctgcgttaccgctgcaatgacaccatcccagaggactatgagacccatcagctgcggcaagcctagggaccagggtgaggacccgccggatgacagccaccctcaccgcggctggatgggggctctgcacccagccccaaggggtggccgtcctgagggggaagtgagaagggctcagagaggacaaaataaagtgtgtgtgcagggaaaaaaaaaaaaaaaaaaaaaaaaaaaaaaaaaaa

SEQ ID NO: 2 - protein, 724 aa, Homo sapiens
MLRELQETNAALQDVRELLRQQVREITFLKNTVMECDACGMQQSVRTGLPSVRPLLHCAPGFCFPGVACIQTESGARCGPCPAGFTGNGSHCTDVNECNAHPCFPRVRCINTSPGFRCEACPPGYSGPTHQGVGLAFAKANKQVCTDINECETGQHNCVPNSVCINTRGSFQCGPCQPGFVGDQASGCQRRAQRFCPDGSPSECHEHADCVLERDGSRSCVCAVGWAGNGILCGRDTDLDGFPDEKLRCPERQCRKDNCVTVPNSGQEDVDRDGIGDACDPDADGDGVPNEKDNCPLVRNPDQRNTDEDKWGDACDNCRSQKNDDQKDTDQDGRGDACDDDIDGDRIRNQADNCPRVPNSDQKDSDGDGIGDACDNCPQKSNPDQADVDHDFVGDACDSDQDQDGDGHQDSRDNCPTVPNSAQEDSDHDGQGDACDDDDDNDGVPDSRDNCRLVPNPGQEDADRDGVGDVCQDDFDADKVVDKIDVCPENAEVTLTDFRAFQTVVLDPEGDAQIDPNWVVLNQGREIVQTMNSDPGLAVGYTAFNGVDFEGTFHVNTVTDDDYAGFIFGYQDSSSFYVVMWKQMEQTYWQANPFRAVAEPGIQLKAVKSSTGPGEQLRNALWHTGDTESQVRLLWKDPRNVGWKDKKSYRWFLQHRPQVGYIRVRFYEGPELVADSNVVLDTTMRGGRLGVFCFSQENIIWANLRYRCNDTIPEDYETHQLRQA

SEQ ID NO: 3 - DNA, 1701 bp, Homo sapiens
cacatgcttccctccaccaaaactgccctcaccttttccctctgctgatccaagtcctccttttcttttatgtctgtctccttgctacctcctccaggaagccctcggtgatttttttgtaggctccccagaaaacatatctggctgtgagtatagattcacccccgccctcgggcagtggccttaggccagtcactttttctctctgggcctcagtttctctgtctatagaatagacgctgtgagtactggaaggtgggagtggagagtgttaactgattgcaggaggttaaggggttttgtaactccagagtgtggctggccagttagcggtaacttttatttttattacaggctgttcccacagcagctggagcacagtttggaaggtatggcacagcctggacaaacagaagccccggacctccccttggtagagccctttaacttgctcccctccagatgggggcctcacaccccattgcgcagattggaaaaccaagtgggcctgtccccttggacaggggttggggcaagatcctgaacgctgtcccctcctccaccagccgagggaccatggggaggggagggaacaccagcaatgagttgggttggggggagtcatttgcagccctccagcgttggggccagaagcggcctccttggacagaggcaggaaaattgagagtcccaggtctcaactgcccctcccctattttccattcatcatcataatcatcattactattaatcattaattaataattattaacttattacctccattgtgcaagggaggaattacgcctgggtaatttttgtacttttagtagagatggggtttcaccatgttagccaggctggtctcaaactcctgacctcaggtgatctgcccgccttggcttcccaaagtgctgggattacaggtgtgagccaccgcacccagcaacttacccagttttgaagcacttcaggaggagtggagggccagtcagtctgatccatagtgggtggacctattttttcagacgctggtgactctgtttcccgaagtgtgagctgagagcgtggccatggagcctgccttgtttggaactggaactcaggtttggcatacagcaagcactcaatcaatcaatcaatcaatgagctgaatgctatggctggatcctgtaatcccagttatgtggggagtatcgcttgaggccgggagtttgagactactagcctgcaggacatagccagacccggtctctaaaaataaaaataaaaataaaaataaattagctggacttggtggtgcgggcttgtagtcccagctacatgggagactgaagcaagaggatgcgatacagccacaccccgtcacacacacacacacacacacacacagacgcacacacacagtgaatgaaagtgggggcagtaccccctgactccctgccccaccagctctctccacagaccccgggactcagtttccccaccatgtcggattcagccgcgggcgacttcgcggggcattccgggcggggacttgaacgcaggggccagcgccatctgtttaccttgaggctggacgttgggcagggctgtggtgggccgtccctggggccggccgtgccttggggataaataggccccgcgggcctcgtgggcggtagaaagcgagcagccacccagctccccgccaccgccatg

SEQ ID NO: 4 - DNA, 2274 bp, Homo sapiens
atggtccccgacaccgcctgcgttcttctgctcaccctggctgccctcggcgcgtccggacagggccagagcccgttgggctcagacctgggcccgcagatgcttcgggaactgcaggaaaccaacgcggcgctgcaggacgtgcgggactggctgcggcagcaggtcagggagatcacgttcctgaaaaacacggtgatggagtgtgacgcgtgcgggatgcagcagtcagtacgcaccggcctacccagcgtgcggcccctgctccactgcgcgcccggcttctgcttccccggcgtggcctgcatccagacggagagcggcggccgctgcggcccctgccccgcgggcttcacgggcaacggctcgcactgcaccgacgtcaacgagtgcaacgcccacccctgcttcccccgagtccgctgtatcaacaccagcccggggttccgctgcgaggcttgcccgccggggtacagcggccccacccaccagggcgtggggctggctttcgccaaggccaacaagcaggtttgcacggacatcaacgagtgtgagaccgggcaacataactgcgtccccaactccgtgtgcatcaacacccggggctccttccagtgcggcccgtgccagcccggcttcgtgggcgaccaggcgtccggctgccagcgcggcgcacagcgcttctgccccgacggctcgcccagcgagtgccacgagcatgcagactgcgtcctagagcgcgatggctcgcggtcgtgcgtgtgtcgcgttggctgggccggcaacgggatcctctgtggtcgcgacactgacctagacggcttcccggacgagaagctgcgctgcccggagccgcagtgccgtaaggacaactgcgtgactgtgcccaactcagggcaggaggatgtggaccgcgatggcatcggagacgcctgcgatccggatgccgacggggacggggtccccaatgaaaaggacaactgcccgctggtgcggaacccagaccagcgcaacacggacgaggacaagtggggcgatgcgtgcgacaactgccggtcccagaagaacgacgaccaaaaggacacagaccaggacggccggggcgatgcgtgcgacgacgacatcgacggcgaccggatccgcaaccaggccgacaactgccctagggtacccaactcagaccagaaggacagtgatggcgatggtataggggatgcctgtgacaactgtccccagaagagcaacccggatcaggcggatgtggaccacgactttgtgggagatgcttgtgacagcgatcaagaccaggatggagacggacatcaggactctcgggacaactgtcccacggtgcctaacagtgcccaggaggactcagaccacgatggccagggtgatgcctgcgacgacgacgacgacaatgacggagtccctgacagtcgggacaactgccgcctggtgcctaaccccggccaggaggacgcggacagggacggcgtgggcgacgtgtgccaggacgactttgatgcagacaaggtggtagacaagatcgacgtgtgtccggagaacgctgaagtcacgctcaccgacttcagggccttccagacagtcgtgctggacccggagggtgacgcgcagattgaccccaactgggtggtgctcaaccagggaagggagatcgtgcagacaatgaacagcgacccaggcctggctgtgggttacactgccttcaatggcgtggacttcgagggcacgttccatgtgaacacggtcacggatgacgactatgcgggcttcatctttggctaccaggacagctccagcttctacgtggtcatgtggaagcagatggagcaaacgtattggcaggcgaaccccttccgtgctgtggccgagcctggcatccaactcaaggctgtgaagtcttccacaggccccggggaacagctgcggaacgctctgtggcatacaggagacacagagtcccaggtgcggctgctgtggaaggacccgcgaaacgtgggttggaaggacaagaagtcctatcgttggttcctgcagcaccggccccaagtgggctacatcagggtgcgattctatgagggccctgagctggtggccgacagcaacgtggtcttggacacaaccatgcggggtggccgcctgggggtcttctgcttctcccaggagaacatcatctgggccaacctgcgttaccgctgcaatgacaccatcccagaggactatgagacccatcagctgcggcaagcctag

SEQ ID NO: 5 - protein, 757 aa, Homo sapiens
MVPDTACVLLLTLAALGASGQGQSPLGSDLGPQMLRELQETNAALQDVRDWLRQQVREITFLKNTVMECDACGMQQSVRTGLPSVRPLLHCAPGFCFPGVACIQTESGGRCGPCPAGFTGNGSHCTDVNECNAHPCFPRVRCINTSPGFRCEACPPGYSGPTHQGVGLAFAKANKQVCTDINECETGQHNCVPNSVCINTRGSFQCGPCQPGFVGDQASGCQRGAQRFCPDGSPSECHEHADCVLERDGSRSCVCRVGWAGNGILCGRDTDLDGFPDEKLRCPEPQCRKDNCVTVPNSGQEDVDRDGIGDACDPDADGDGVPNEKDNCPLVRNPDQRNTDEDKWGDACDNCRSQKNDDQKDTDQDGRGDACDDDIDGDRIRNQADNCPRVPNSDQKDSDGDGIGDACDNCPQKSNPDQADVDHDFVGDACDSDQDQDGDGHQDSRDNCPTVPNSAQEDSDHDGQGDACDDDDDNDGVPDSRDNCRLVPNPGQEDADRDGVGDVCQDDFDADKVVDKIDVCPENAEVTLTDFRAFQTVVLDPEGDAQIDPNWVVLNQGREIVQTMNSDPGLAVGYTAFNGVDFEGTFHVNTVTDDDYAGFIFGYQDSSSFYVVMWKQMEQTYWQANPFRAVAEPGIQLKAVKSSTGPGEQLRNALWHTGDTESQVRLLWKDPRNVGWKDKKSYRWFLQHRPQVGYIRVRFYEGPELVADSNVVLDTTMRGGRLGVFCFSQENIIWANLRYRCNDTIPEDYETHQLRQA

SEQ ID NO: 6 - DNA, 57 bp, Homo sapiens
gacaactgcccgctggtgcggaacccagaccagcgcaacacgtacgaggacaagtgg

SEQ ID NO: 7 - protein, 19 aa, Homo sapiens
DNCPLVRNPDQRNTYEDKW

SEQ ID NO: 8 - DNA, 8923 bp, Mus musculus
  modified_base (3840)..(8763): n = a, c, g or t/u
gaattctgtttgtctgaaggtgcggggaggggggcggaggaaccctcacaacctcctgctgacctgggctgagtcacacccataggtcccttttgcaggactttgaagtgcggagggagtgtgggagccagagtgggtgagttgggaccttttgaatacttatgactttgtttccccaatgctatctacggggacagatgaagattgtcttgcatacaacaagcactcagtggtgaaccagagtgagtggagagccccaatttactcttcaaggctcatgcctttctctcctcagaccccaccatatactgatcagctgcgggtcatcccgcagggctgtggggcgcggccctgaatgcagagcccggcctgtttaccttgtggcgggctctgggcagggccgcgggggctgtcccggacccggcgcggggataaataggccgcgggctcgaaggcgcagacagcagctgcagctccgccgccatgggccccactgcctgcgttctagtgctcgccctggctatcctgcgggcgacaggccagggccagatcccgctgggtaaagccgcttagtaggggacatggttggacaggaggcccctccaggctcatgattcttgctcctcagaacttggggtctgctctccaggaacgtccggggttcctgaaaaatgaagcggcgggtggagcttggatggccccggaggtggcgggaggggtgatggagtggttgggtcgcccatcactggcccctgtggacgcaggtggagacctggccccacagatgctgcgagaacttcaggagactaatgcggcgctgcaagacgtgagagagctgttgcgacagcaggtaaggaaacagagacagcagcgtggagacagaacagggacaggggcatacagagaggactaagaatggtagagaaccgagagagagagagagagaaaggcagcgcggggcagaacaggtgcagaaggcacatcgcagtgacgagcggggaacagaagggacagaaaaaggacagagaggaaaaacagagcagggaagagaggacagggcgtgcggggagcagaagggacagaaaaggacagagaggaaaaacagagcagggaagagaggacagggcgtgggacagagaggatggaaacaggggcagggacaggattctgggactagatgcacagagtcagagacagggagacagggactctggaatgggtttgtgcaaaggcggatgtaaagggggtggttgtgggcaccgggcttcccaggaagaggggtcgtgggaagaggagggggaccgagaggaggcgcatgggaaggggtcccgctgacttcctgcccggcaggtcaaggagatcaccttcctgaagaatacggtgatggaatgtgatgcttgcggtgagcatagaccggacccaagggcaggaggaagggtgggggcacagagcgaaacggaggaaaagggctggggaaggaagctggcgggggcgaaagcagacagcgacagagcggactggacgacggagagaaggcctgggagatgcgcgaagagcaacgacagggctggcaggaggaccgcacggccggagggcgcgcgctcggacctgcccagtcccagcttgggctaaaccgaaccccagaggggcgtggtcctgagcggccccaccccgggaggggtcaagcccatcttgtgggcggggccggacaggcgcctcctctgggcgtggcccaggttggctcctctccggggcgtggcctaacttctgcgggtccgcaggaatgcagcccgcacgcactccaggcctgagcgtgcggccagtgccgctctgcgcacccggctcctgcttcccggcgtagtctgctccgagacagctacgggcgcgcgctgcggcccctgccctcctggctacaccggcaacggctcgcactgcaccgacgttaatgaggtgcgcgggctcttcacacccccgccgtcctgtccctacccgggccgaccccaccacaaattccctccatccgacgacgcccccctcatccactgagggtgttctcctggaccagcccctcgcagcccggggctccaaaccagaagacctcacgtctaacagggggggcccacccaggccaactccatcactctctcagacacacactctgaccactccttttgtccctaccgacgcccaccccaacgaccacttgggcgccgggggtgtctctctccaaccctcctcaccactgggacgtcctcgaccggaccacgtgtctactttagagagtcgccctccccgacggggcaatcgccgccgtgcctgcacgcccaggcggccacttcctccctcggctggggatgcccgccccacaaattcatttcctcatccctaagaggtcacaactccatgcccataaaggcaaagtcgtcagcgaccctcgggttctctatcccgtgtggcaccctatttcaagagctggctgaagatggcccttaagcgccctggaatgcagacgcatgccaatctgcattctctgcattgagttcgagcccagcctggtatacatggtgtgttccagagcaggcagagctaagcagtgagatcttgtctccaacaaaacaaacccctgtccacatccgggaagccccaaggtgcggctctggcggtccagcttggggcctctaatcctgtgtgcttctttctcacagtgcaatgctcacccctgtttcccgcgggtgcggtgcatcaataccagccctggctttactgcgaagcctgtccccctgggttcagcggacccacccacgagggcgtgggactgaccttcgctaagtccaacaaacaagtaagggaagctggggactctacatttatggcagaagggaatgaaaggcattttgtccagaaaactcactccaaagagaaagttcttgcagcgggggtggggtggtggacaggttgcaaagaggcgtgacccatgggaaacgtgtgttgtctgcccgttgtctccatccttcaggtttgcacggatattaatgagtgtgagaccgggcagcacaattgcgttcccaactccgtgtgcgtcaacacccgggtaaggaaggcagggatggtgaggttgaccccatcaaggcccaaatgggtgacccgctgactgcctgcgcctgcacctagggctccttccagtgcggcccctgccagcccggtttcgtgggcgaccagacgtcaggctgccagcggcgtgggcagcacttctgccccgatgggtcacccagcccgtgccatgagaaagcaaactgcgtcctggagcgggatggctcgaggtcttgcgtggtgagtgcagaagcaaaggtcgtggaagaggggtcccggagctccggcgtacgtggacatttccaccgtctcccctatgcagtgtgcagtcggctgggccggcaacgggctcctgtgcggccgcgacacggacctggacggttttcctgacgagaagcttcgctgctcagagcgccagtgtcgcaaggtgggcgtgaccaggagggcgtgaccgggagggtgtggtgagcacggtagacacgagccttaccccaccctaccccccatccctgcttcccaggacaactgcgtgacggtgcccaattcggggcaggaggatgtggaccgggacggcatcggagatgcttgtgacccggatgcggacggggatggagtccctaacgagcaagtaaggctgtgtaggatcgtccgtgggcaggacttggtggcagcgtgacctctaaggtcacgctagttatctagcttccagcagagggaccagaccttcttggagatgggctggtctgaaanatgggtctttaaaacttatttatttatgtatttatttgtgggtatttatttgtgggtatgtgcacgcacgtgtgttctcgcgagtgtgacacagtgtggaagactaagggttacttgcaggagtctgttctctctttacatcgtaagctccgggaagacagaactttttctacttccctgtctggcaactaggacatgatctctgtctcatgctgactttgccttctacttgccctgccttctaggacaattgcccgctggttcgaaacccagaccagcgtaactcggacagtgataagtggggagatgcctgcgacaactgccggtccaagaagaatgacgatcagaaagatacagacctggatggccggggcgatgcctgcgacgacgacatagatggcgacggtgagcatggctggggagtgaagggtggaacccatctctcagtgaactgcaaggcttggaactgagtggtggcttggtctagagtgccctggtgtggctaaagtcaagcagagggaaacacgaagccaggtctgggagagaagagggctgggacatgaggggtggggtacccagtttaacatcccttgtgggtttcaacatgatgcatagcagggaatggcctagaactcctgatcttcctgccttcgcctactgagtattgggaccgacaggtgtgcacaactgtgtcctgcttgaccatccttttttcttatcttttatgtatgtgaatacattgttgctgttgctatcttcagacactccagaaagaaggcatttgatccctttacagatggttgtggccaccatgtagttgctgggaattgaactcaggacctctggaagagcagccagtgctcttaagcgctgagccatctctccagcccttggccatccttttttatttgacattattgttgttactgtttttgagacagggtctctctccgtagccccagcggtcttgaaacacactctatagaccaagctggcttaagactcacagagatctgactgtctctgctttctgagtgtgggattaaaggaatgtgccactatgcctcactttaattttttttcatgaacttatttttagtatgctttagtgcaatttttagtatgctttagtatgcttcaggctcttctaagccttcctcggcccctgtgcccctcttttcactcccacctcagcaccctaagtctccccccaagattagttgcatttctgatctcctgcctgctaccccaggcccattcagggtagaggccaatgaccactctgcccaagatcttacatggctgctggttctttctctctctgaagcacagtaaattctttcctcactttattttttttttaaatcttgttttatttgatttttttcccccaagacagggtctcactgtgtagtttgatctgttctagaactcactctgtagagtaggctggccttgaactcacagagatttgcctgcctctgcctccagagtgccaacaggcccagcaactttgtaatgtaactgatctatcctgtgtccatgctcctgtgtatgcatgtgtgtgcaagagtggtatgacacacacatggaggtcagagggctgcctgcattgcaagagtgagttctctctcaccacaccagccccagagggcaatctcagaccccttggtcatcgaccccttatgatctgtgttgggacggtaccatcctagcctggccctagtctcaggatcctaccttcgttctctgatttacctcaggatacgaaatgtagctgacaactgtccccgggtgcccaactttgaccagagtgacagtgatggtgatggtgttggggatgcctgtgacaactgtccccagaaagataacccagaccaggtgggccactttctatgtgcactttagtttggggagcataatggatcctgccaagggcattctgagggtgggggttcttggggtgggaaggacctggctgtggagttggaatgggaatgactactgagtacctagccctgactgtgacccttgatgccattccagagggatgtggaccacgactttgtgggtgatgcctgtgatagtgaccaagaccagtaaggagcccttgggaaggtagcaatggaatattgcatgacaacccccttccagagtctcacgtcccatttccacactctagggatggggatggtcaccaggactcccgggacaactgccccacagtacccaacagtgcccagcaggactcagatcatgatggcaagggcgatgcctgtgatgacgatgatgacaatgacggagttcctgatagccgggacaactgccgcttggtgcctaaccctggccaagaggacaatgaccgtaaggatggagtgatcgtgattattagctggtgtggtctctggtgtggacttggtcagtaacagatgtgggtgtggccagcagctggtaggaggaggcagaggtgcctggtgtgggcgtggtcagcagttagcataggtggaggggggtgctgagctgagccctaccttctttcaggggatggcgtgggtgacgcgtgtcagggtgacttcgatgctgacaaggttatagacaagatcgatgtgtgccccgagaacgccgaggtcaccctcaccgacttcagggccttccagacggttgtgttggaccccgagggtgatgcgcagatcgatcccaactgggtggtgctcaatcaggtgtggctagggctggggtagcggtctaggggggcccaggtgccgcctcagcaagacctccaccactcggcgctggcctgagccccttgttcttctgacctcaccagggaatggagatcgttcagaccatgaacagtgaccctggcctggctgtgggtgaggcggggcagggctatggggcgtgatcacggagggcttggccactctaatcatgggaagagtagggctaagggggttaggacaaatggcagtttgtattgagtggtcataggtgggtgggtcataggccatggagagacggggctggttggtcaggatctaggaagggctgggtggggcctttggggcattgctgtgggacgtgtgacccctgagagctagggattggaagtgtctgaggatgtggccgatgctatgttggggtgtggccttgtttggagaagcaggtcttgtttggagggtcagggcctgactctgaggtgtccagagcaagcatgcttctggagacccccttcctctcccctcctacaggttacacagccttcaacggcgtggacttcgagggcacattccatgtaaacaccgccactgatgatgactatgctggtttcatctttggctaccaagacagctccagtttctatgtagtcatgtggaaacagatggagcagacgtactggcaggccaatcccttccgggctgtggctgagccagggattcagctcaaggtgctggctgggctgtgcccacacacattatatactcttcagccttcaccgccaatgccttcgtagccctccagcattgtcccatgcccctcaaagttgtcaccactccttattctcgtgcccagcccccaccctcccaccaccattgccacggggttaaatcctctcagacccataaataccttctctggagggtcagagaagacactgctttgttacagtgcttggggcacactcaagggaactggagtttggacccccctgaacccacctaaatgctgggtatggatggtgatccacctgtaattccagccttgaaaggtagaaacaggatccctagatatactagccacactgggaaaccccaggccttatggaacaggatagaaaagctacttaaggatgactccaacagcttctgcccttcgtgcacatgtgcatgcacccacacaggttcagaaacgtgcatactcagagagaaaaaaattgaagatagaccctgtctcaaaaaaaaaaaaaaaggaaaaagaaaaaaagaaaagaaaagaaaagaaaaaagaaaagaaaaaaaaagggggggggaactactccttgggatcatcacatgtccctttgggctcccaggcctgtcacagagccctagatttccctggacaccccagatcctcctatagacacctcacaacaggtccccaggtctgtagaccccagtttatcccagcaggctgctgcctcctaataaggagccaggccctctagatcccctaaggtatccctgtatttccagaattcacacagactcctggctccactacccacagtggtccagccaagagctctcacttgccctcttgaagcatggaccttccctatactccaaccactcataactcccacctgatctaccagacagggctctgatctgtttcctctcctgcctgtggcaggctgtcaagtcctctacaggtcccggggaacagctccgaaacgcactttggcacacgggggacacagcatcccaggtgcggctgctgtggaaggatcctcgaaacgtgggctggaaggataaaacatcctaccgctggttcctgcagcaccggcctcaagttggctacatcaggtgggcacggccctgctgctgctgagctgtgctttgctgctgctccaggagaaacgggctccgtttacagtacatcatggtcttacggggagatgccagaaccccaatacctcctatgtacagggcacctgacatacattctcacagagggaaactgaggcaggggccccaacaccagcccttattttgagtgggaactaaanatgaagggggtggcaagcagggaacccaacctcaatagtcctttatcacagggtgcggttctatgagggtcctgagctagtagctgacagcaatgtggtgttgcacacggccatgcgtggtggccgcctgggtgtcttctgcttctcccaanagaacatcatctgggctaacctgcgctaccgttgcaatggtgagcgagaggccagcgggctggacccaaaaggctccagaaacctctctcacctgttgccttccaatctgcagatacaatccctgaggactacgagagtcaccggctgcagagagtctag

SEQ ID NO: 9 - DNA, 8524 bp, Mus musculus
  modified_base (3651)..(3900): n = a, c, g or t/u
gcagctccgccgccatgggccccactgcctgcgttctagtgctcgccctggctatcctgcgggcgacaggccagggccagatcccgctgggtaaagccgcttagtaggggacatggttggacaggaggcccctccaggctcatgattcttgctcctcagaacttggggtctgctctccaggaacgtccggggttcctgaaaaatgaagcggcgggtggagcttggatggccccggaggtggcgggaggggtgatggagtggttgggtcgcccatcactggcccctgtggacgcaggtggagacctggccccacagatgctgcgagaacttcaggagactaatgcggcgctgcaagacgtgagagagctgttgcgacacgaggtaaggaaacagagacagcagcgtggagacagaacagggacaggggcatacagagaggactaagaatggtagagaaccgagagagagagagagagaaaggcagcgcggggcagaacaggtgcagaaggcacatcgcagtgacgagcggggaacagaagggacagaaaaggacagagaggaaaaacagagcagggaagagaggacagggcgtgcggggagcagaagggacagaaaaggacagagaggaaaaacagagcagggaagagaggacagggcgtgggacagagaggatggaaacaggggcagggacaggattctgggactagatgcacagagtcagagacagggagacagggactctggaatgggtttgtgcaaaggcggatgtaaagggggtggttgtgggcaccgggcttccaggaagaggggtcgtgggaagaggagggggaccgagaggaggcgcatgggaaggggtcccgctgacttcctgcccggcaggtcaaggagatcaccttcctgaagaatacggtgatggaatgtgatgcttgcggtgagcatagaccggacccaagggcaggaggaagggtgggggcacagagcgaaacggaggaaaagggctggggaaggaagctggcgggggcgaaagcagacagcgacagagcggactggacgacggagagaaggcctgggagatgcgcgaagagcaacgacagggctggcaggaggaccgcacggccggagggcgcgcgctcggacctgcccagtcccagcttgggctaaaccgaaccccagaggggcgtggtcctgagcggccccaccccgggaggggtcaagcccatattgtgggcggggccggacaggcgcctgcctctgggcgtggcccaggttggctcctctccggggcgtggcctaacttctgcgggtccgcaggaatgcagcccgcacgcactccaggcctgagcgtgcggccagtgccgctctgcgcacccggctcctgcttccccggcgtagtctgctccgagacagctacgggcgcgcgctgcggcccctgccctcctggctacaccggcaacggctcgcactgcaccgacgttaatgaggtgcgcgggctcttcacacccccgccgtcctgtccctacccgggccgaccccaccacaaattccctccatccgacgacgcccccctcatccactgagggtgttctcctggaccagcccctcgcagcccggggctccaaaccagaagacctcacgtctaacagggggggcccacccaggccaactccatcactctctcagacacacactctgaccactccttttgtccctaccgacgcccaccccaacgaccacttgggcgccgggggtgtctctctccaaccctcctcaccactgggacgtcctcgaccggaccacgtgtctactttagagagtcgccctccccgacggggcaatcgccgccgtgcctgcacgcccaggcggccacttcctccctcggctggggatgcccgccccacaaattcatttcctcatccctaagaggtcacaacttccatgcccataaaggcaaagtcgtcagcgaccctcgggttctctatcccgtgtggcaccctatttcaagagctggctgaagatggcccttaagcgccctggaatgcagacgcatgccaatctgcattctctgcattgagttcgagcccagcctggtatacatgatgtgttccagagcaggcagagctaagcagtgagatcttgtctccaacaaaacaaacccctgtccacatccgggaagccccaaggtgcggctctggcggtccagcttggggcctctaatcctgtgtgcttctttctcacagtgcaatgctcacccctgtttcccgcgggtgcggtgcatcaataccagccctggctttcactgcgaagcctgtccccctgggttcagcggacccacccacgagggcgtgggactgaccttcgctaagtccaacaaacaagtaagggaagctggggactctacatttatggcagaagggaatgaaaggcattttgtccagaaaactcactccaaagagaaagttcttgcagcgggggtggggtggtggacaggttgcaaagaggcgtgacccatgggaaacgtgtgttgtctgcccgttgtctccatccttcaggtttgcacggatattaatgagtgtgagaccgggcagcacaattgcgttcccaactccgtgtgcgtcaacacccgggtaaggaaggcagggatggtgaggttgaccccatcaaggcccaaatgggtgacccgctgactgcctgcgcctgcacctagggctccttccagtgcggcccctgccagcccggtttcgtgggcgaccagacgtcaggctgccagcggcgtgggcagcacttctgccccgatgggtcacccagcccgtgccatgagaaagcaaactgcgtcctggagcgggatggctcgaggtcttgcgtggtgagtgcagaagcaaaggtcgtggaagaggggtcccggatctccggcgtacgtggacatttccaccgtctcccctatgcagtgtgcagtcggctgggccggcaacgggctcctgtgcggccgcgacacggacctggacggttttcctgacgagaagcttcgctgctcagagcgccagtgtcgcaaggtgggcgtgaccaggagggcgtgaccgggagggtgtggtgagcacggtagacacgagccttaccccaccctaccccccatccctgcttcccaggacaactgcgtgacggtgcccaattcggggcaggaggatgtggaccgggacggcatcggagatgcttgtgacccggatgcggacggggatggagtccctaacgagcaagtaaggctgtgtaggatcgtccgtgggcaggacttggtggcagcgtgacctctaaggtcacgctagttatctagcttccagcagagggaccagaccttcttggagatgggctggtctgaaaaatgggtctttaaaacttatttatttatgtatttatttgtgggtatttatttgtgggtatgtgcacgcacgtgtgttctcgcgagtgtgacacagtgtggaagactaagggttacttgcaggagtctgttctctctttacatcgtaagctccgggaagacagaactttttctacttccctgtctggcaactaggacatgatctctgtctcatgctgactttgccttctacttgccctgccttctaggacaattgcccgctggttcgaaacccagaccancgtaactcggacagtgataagtggggagatgcctgcgacaactgccggtccaagaagaatgacgatcagaaagatacagacctggatggccggggcgatgcctgcgacgacgacatagatggcgaccgtgagcatggctggggagtgaagggtggaacccatctctcagtgaactgcaaggcttggaactgagtggtggcttggtctagagtgccctggtgtggctaaagtcaagcagagggaaacacngaagccaggtctgggagagaagagggctgggacatgaggggtggggtacccagtttaacatcccttgtgggtttcaacatgatgcatagcagggaatggcctagaacccctgatcttcctgccttcgcctactgagtattgggaccgacaggtgtgcacaactgtgtcctgcttgaccatccttttttcttatcttttatgtatgtgaatacattgttgctgttgctatcttcagacactccagaagaaggcatttgatccctttacagatggttgtggccaccatgtagtcgctgggaattgaactcagacctctgaaagagcagccagtctcttaagcgttgagccatctctccagcccttggccatccttttttatttgacattattgttattactgtttttgggacagggtctctctcggtagccccagcggtcttgaaacacactctatagaccaagctggcttaagactcacagagatctgactgtctctgctttctgagtgtgggattaaaggaatgtgccactatgcctcactttaattttttttcatgaacttatttttagtatgctttagtgcaatttttagtatgctttagtatgcttcaggctcttataagccttcctcggcccctgtgcccctcttttcactcccacctcagcaccctaagtctccccccaagattagttgcatttctgatctcctgcctgctaccccaggcccattcagggtagaggccaatgaccactctgcccaagatcttacatggctgctggttctttctctctctgaagcacagtaaactctttcctcactttatttttttttttaaatcttgttttatttgatttttttccccaagacagggtctcactgtgtagtttgatctgttctagaactcactctgtagagtaggctggccttgaactcacagagatttgcctgcctctgcctccagagtgccaacaggcccagcaactttgtaatgtaactgatctatcctgtgtccatgctcctgtgtatgcatgtgtgtgcaagagtggtatgacacacacatggaggtcagagggctgcctgcattgcaagagtgagttctctctcaccacaccagccccagagggcaatctcagaccccttggtcatcgaccccttatgatctgtgttgggacggtaccatcctagcctggccctagtctcaggatcctaccttcgttctctgatttacctcaggaatacgaaatgtagctgacaactgtccccgggtgcccaactttgaccagagtgacagtgatggtgatggtgttggggatgcctgtgacaactgtccccagaaagataacccagaccaggtgggccactttctatgtgcactttagtttggggagcataatggatcctgccaagggcattctgagggtgggggttcttggggtgggaaggacctggctgtggagttggaatgggaatgactactgagtacctagccctgactgtgacccttgatgccattccagagggatgtggaccacgactttgtgggtgatgcctgtgatagtgaccaagaccagtaaggagcccttgggaaggtagcaatggaatattgcatgacaacccccttccagagtctcacgtccatttccacactctagggatggggatggtcaccaggactcccgggacaactgccccacagtacccaacagtgcccagcaggactcagatcatgatggcaagggcgatgcctgtgatgacgatgatgacaatgacggagttcctgatagccgggacaactgccgcttggtgcctaaccctggccaagaggacaatgaccgtaaggatggagtgatcgtgattattagctggtgtggtctctggtgtggacttggtcagtaacagatgtgggtgtggccagcagctggtaggaggaggcagaggtgcctggtgtgggcgtggtcagcagttagcataggtggaggggggtgctgagctgagccctaccttctttcaggggatggcgtgggtgacgcgtgtcagggtgacttcgatgctgacaaggttatagacaagatcgatgtgtgccccgagaacgccgaggtcaccctcaccgacttcagggccttccagacggttgtgttggaccccgagggtgatgcgcagatcgatcccaactgggtggtgctcaatcaggtgtggctagggctggggtagcggtctaggggggcccaggtgccgcctcagcaagacctccaccactcggcgctggcctgagccccttgttcttctgacctccacagggaatggagatcgttcagaccatgaacagtgaccctggcctggctgtgggtgaggcggggcagggctatggggcgtgatcacggagggcttggccactctaatcatgggaagagtagggctaagggggttaggacaaatggcagtttgtattgagtggtcataggtgggtgggtcataggccatggagagacggggctggttggtcaggatctaggaagggctgggtggggcctttggggcattgctgtgggacgtgtgacccctgagagctagggattggaagtgtctgaggatgtggccgatgctatgttggggtgtggccttgtttggagaagcaggtcttgtttggagggtcagggcctgactctgaggtgtccagagcaagcatgcttctggagacccccttcctctcccctcctacaggttacacagccttcaacggcgtggacttcgagggcacattccatgtaaacaccgccactgatgatgactatgctggtttcatctttggctaccaagacagctccagtttctatgtagtcatgtggaaacagatggagcagacgtactggcaggccaatcccttccgggctgtggctgagccagggattcagctcaaggtgctggctgggctgtgcccacacacattatatactcttcagccttcaccgccaatgccttcgtagccctccagcattgtcccatgcccctcaaagttgtcaccactcctcattctcgtgcccagcccccaccctcccaccaccattgccacggggttaaatcctctcagacccataaataccttctctggagggtcagagaagacactgctttgttacagtgcttggggcacactcaagggaactggagtttggacccccctgaacccacctaaatgctgggtatggatggtgatccacctgtaattccagccttgaaaggtagaacaggatccctagatatactagccacactgggaaaccccaggccttatggaacaggatagaaaagctacttaaggatgactccaacagcttctgcccttcgtgcacatgtgcatgcacccacacaggttcagaaacgtgcatactcagagagaaaaaaattgaagatagaccctgtctcaaaaaaaaaaaaaagaaaaagaaaaaaagaaaagaaaagaaaagaaaaaagaaaagaaaaaaaaagggggggggaactactccttgggatcatcacatgtccctttgggctcccaggcctgtcacagagccctagatttccctggacaccccagatcctcctatagacacctcacaacaggtccccaggtctgtagaccccagtttatcccagcaggctgctgcctcctaataaggagccaggccctctaaatcccctaaggtatccctgtatttccagaattcacacagactcctggctccactacccacagtggtccagccaagagctctcacttgccctcttgaagcaaggaccttccctatactccaaccactcataactcccacctgatctaccagacagggctctgatctgtttcctctcctgcctgtggcaggctgtcaagtcctctacaggtcccggggaacagctccgaaacgcactttggcacacgggggacacagcatcccaggtgcggctgctgtggaaggatcctcgaaacgtgggctggaaggataaaacatcctaccgctggttcctgcagcaccggcctcaagttggctacatcaggtgggcacggccctgctgctgctgagctgtgctttgctgctgctccaggagaaacgggctccgtttacagtacatcatggtcttacggggagatgccagaaccccaatacctcctatgtacagggcacctgacatacattctcacagagggaaactgaggcaggggccccaacaccagcccttattttgagtgggaactaaagatgaagggggtggcaagcagggaacccaacctcaatagtcctttatcacagggtgcgggtctatgagggtcctgagctagtagctgacagcaatgtggtgttggacacggccatgcgtggtggccgcctgggtgtcttctgcttctcccaagagaacatcatctgggctaacctgcgctaccgttgcaatggtgagcgagaggccagcgggctggacccaaaaggctccagaaacctctctcacctgttgccttccaatctgcagatacaatccctgaggactacgagagtcaccggctgcagagagtttagggaccagtggggtcccgctgcctgatggactgtggtggcataagctacgggtgtgtgtgtgtgtggggtctgg

SEQ ID NO: 10 - protein, 755 aa, Mus musculus
  MOD_RES (334): xaa = anything
MGPTACVLVLALAILRATGQGQIPLGGDLAPQMLRELQETNAALQDVRELLRHEVKEITFLKNTVMECDACGMQPARTPGLSVRPVPLCAPGSCFPGVVCSETATGARCGPCPPGYTGNGSHCTDVNECNAHPCFPRVRCINTSPGFHCEACPPGFSGPTHEGVGLTFAKSNKQVCTDINECETGQHNCVPNSVCVNTRGSFQCGPCQPGFVGDQTSGCQRRGQHFCPDGSPSPCHEKANCVLERDGSRSCVCAVGWAGNGLLCGRDTDLDGFPDEKLRCSERQCRKDNCVTVPNSGQEDVDRDGIGDACDPDADGDGVPNEQDNCPLVRNPDXRNSDSDKWGDACDNCRSKKNDDQKDTDLDGRGDACDDDIDGDRIRNVADNCPRVPNFDQSDSDGDGVGDACDNCPQKDNPDQRDVDHDFVGDACDSDQDQDGDGHQDSRDNCPTVPNSAQQDSDHDGKGDACDDDDDNDGVPDSRDNCRLVPNPGQEDNDRDGVGDACQGDFDADKVIDKIDVCPENAEVTLTDFRAFQTVVLDPEGDAQIDPNWVVLNQGMEIVQTMNSDPGLAVGYTAFNGVDFEGTFHVNTATDDDYAGFIFGYQDSSSFYVVMWKQMEQTYWQANPFRAVAEPGIQLKAVKSSTGPGEQLRNALWHTGDTASQVRLLWKDPRNVGWKDKTSYRWFLQHRPQVGYIRVRVYEGPELVADSNVVLDTAMRGGRLGVFCFSQENIIWANLRYRCNDTIPEDYESHRLQRV

SEQ ID NO: 11 - DNA, 2421 bp, Rattus norvegicus
gtcgacatgagccccactgcctgcgttctagtgctcgccctggctgccttgcgggctaccggccagggccagatcccgctgggtggagacctagccccacagatgcttcgagaactccaggagactaatgcggcgctgcaagacgtgagagagctcttgcgacacagggtcaaggagatcaccttcctgaagaatacggtgatggaatgtgacgcttgcggaatgcagcccgcacgcacccccggtctgagcgtgcggccagtcgcgctctgcgcacccggctcctgcttccctggcgtagtctgcacggagacagctaccggcgcgcgctgcggcccctgccctccgggctacaccggcaacggctcgcactgcaccgacgttaatgagtgcaacgctcacccctgtttcccgcgcgtgcggtgcatcaataccagccctggctttcactgcgaagcctgtccccctgggttcagcgggcccacccacgagggtgtggggctgaccttcgccaagaccaacaaacaagtttgcacagatattaatgagtgtgagaccgggcagcacaattgcgttcccaactccgtgtgcgtcaacacccggggctccttccagtgcggtccctgccagcccggcttcgtgggcgaccagaggtcaggctgccagcggcgtgggcaacacttctgccccgacgggtcacccagcccgtgccatgagaaagcagactgtattttggagcgcgacggctcaaggtcctgcgtgtgtgcggtcggctgggccggcaacgggctcctgtgcggacgcgacacagacctggacggtttcccggacgagaagcttcgctgctcagagcgccagtgccgcaaggacaactgcgtgacggtgcccaattcagggcaggaggatgtggaccgggaccgcattggagatgcttgtgacccggatgcggacggggatggagtccctaatgagcaagacaattgcccgctggttcgaaacccagaccagcgcaactccgataaagacaagtggggagatgcctgcgacaactgccggtcccagaagaatgatgaccagaaagatacagaccgggatggccagggcgatgcctgcgacgacgacatagatggcgatcgaatccgaaatgtagctgacaactgtccccgggtgcccaactttgaccagagtgacagcgatggtgatggtgttggggatgcctgtgacaattgtccccagaaagacaacccggaccagagggacgtggaccacgactttgtgggtgatgcctgtgacagtgaccaagaccaggacggggatggacaccaagactcccgggacaactgccccacagtgcccaacagtgcccagcaggactcagaccatgatggcaagggtgatgcctgtgatgatgacgacgacaatgacggagtccctgacagccgggacaattgccgcttggtgcccaacccgggccaagaggacaatgaccgggatggcgtgggtgacgcttgtcagggtgacttcgatgctgacaaggttatagacaagatcgatgtgtgccccgagaacgccgaggtcactctcaccgacttcagggccttccaaacagttgtgctggaccccgagggtgatgcgcagatcgaccccaactgggtggtgctcaatcagggaatggagatcgttcagaccatgaacagtgaccctggcctggctgtgggttacacggcattcaacggtgtagattttgagggaacgttccatgtaaacaccgccaccgatgatgactacgctggcttcatcttcggctatcaagacagctcaagtttctatgtggtcatgtggaaacagatggagcagacgtactggcaggccaatcctttccgggcagtggctgaaccagggattcagctcaaggctgtcaagtcctctacaggtcccggggaacagctccgaaatgcgttgtggcacacgggggacacagcatcccaggtgcggctgctgtggaaggatcctcgaaatgtgggctggaaggataagacatcctaccgctggttcctgcagcaccggcctcaagtcggctacatcagggtgcggttctatgagggtcctgagctagtagctgacagcaacgtggtgctggacacagctatgcgtggtggccgcctgggtgtcttctgcttctcccaagagaatatcatctgggctaacctgcgctaccgttgcaatgatacaatccctgaggactatgagcgtcaccggctgcggagggcctagggaccctaagaggggccccgctgaccgatggactgcggtagcatcggccacaggtgtctgggggggggggtctggcatctttctgaagggatgtctggcctggggaggaaaggcaaataaagaatgtatgtgggggaaaaaaaaaaa

SEQ ID NO: 12 - protein, 755 aa, Rattus norvegicus
MSPTACVLVLALAALRATGQGQIPLGGDLAPQMLRELQETNAALQDVRELLRHRVKEITFLKNTVMECDACGMQPARTPGLSVRPVALCAPGSCFPGVVCTETATGARCGPCPPGYTGNGSHCTDVNECNAHPCFPRVRCINTSPGFHCEACPPGFSGPTHEGVGLTFAKTNKQVCTDINECETGQHNCVPNSVCVNTRGSFQCGPCQPGFVGDQRSGCQRRGQHFCPDGSPSPCHEKADCILERDGSRSCVCAVGWAGNGLLCGRDTDLDGFPDEKLRCSERQCRKDNCVTVPNSGQEDVDRDRIGDACDPDADGDGVPNEQDNCPLVRNPDQRNSDKDKWGDACDNCRSQKNDDQKDTDRDGQGDACDDDIDGDRIRNVADNCPRVPNFDQSDSDGDGVGDACDNCPQKDNPDQRDVDHDFVGDACDSDQDQDGDGHQDSRDNCPTVPNSAQQDSDHDGKGDACDDDDDNDGVPDSRDNCRLVPNPGQEDNDRDGVGDACQGDFDADKVIDKIDVCPENAEVTLTDFRAFQTVVLDPEGDAQIDPNWVVLNQGMEIVQTMNSDPGLAVGYTAFNGVDFEGTFHVNTATDDDYAGFIFGYQDSSSFYVVMWKQMEQTYWQANPFRAVAEPGIQLKAVKSSTGPGEQLRNALWHTGDTASQVRLLWKDPRNVGWKDKTSYRWFLQHRPQVGYIRVRFYEGPELVADSNVVLDTAMRGGRLGVFCFSQENIIWANLRYRCNDTIPEDYERHRLRRA

SEQ ID NO: 13 - DNA, 2302 bp, Equus caballus
agagcgcgccgccgtccagctccccgccgccgccatggttctctccgccgcccccgttctcctgctcgccctggccgccctcgtgtccagccaggggcagaccccgctgggtacagaactgggcccacagatgctgcgcgaactgcaagagaccaacgcggcgctgcaggacgtgcgggagctgctgcggcagcaggtcaaggagatcacgttcctgaaaaacacggtgatggagtgtgacgcgtgcgggatgcagcctgcgcgcaccccccgcgtgagcgtgcggcccctagcccagtgcgcgccgggctcctgcttccctggcgtggcttgtacccagacggcgagcggcgcgcgctgcggaccctgccccgcgggcttcacgggcaacggcccatactgtgccgacgtcaacgagtgcaacgccaatccctgcttccctcgcgtccgctgcatcaataccagccccggtttccgctgcgaggcttgcccgcccgggtacagcggccccacccacgagggcgtggggatggcctttgccaaggccaacaagcaggtttgcacggatattgacgagtgtgagaccgggcagcataactgcgtccccaactccgtgtgcatcaacacccagggctccttccagtgcggcccgtgccagcccggcttcgtaggcgaccaggcatcaggctgccgtccgcgcgcacagcgcttctgccccgacggcacgcccagcccgtgccacgagaaggccgactgcgtcctggagcgcgatggctcgcgatcgtgcgtgtgcgccgtcggctgggccggcaacgggctcctgtgtggccgcgacacggacttggacggcttcccggacgagaagctgcgctgctcggagcgccagtgtcgcaaggataactgcgtgacggtacccaactcaggacaggaggacgcggatcgcgacggcatcggagacgcctgcgacacggacgccgacggagacggagtccccaacgagggggacaactgcccgctggtgcggaacccagaccagcgtaacacggacggcgacaagtggggcgatgcatgcgacaactgccggtcccagaagaacgatgaccagaaggacacagatcaggacggccgaggcgacgcctgcgacgatgacatcgacggcgaccggatccgaaatgcggtggacaactgccccagggtgcccaactcagaccagaaagacagtgatggcgatggtataggggatgtctgtgacaactgtccccagaagagcaacccagaccagagggacgtggaccacgacttcgtgggagacgcttgtgacagcgaccaagacaaggatggggatgggcaccaggactctcgggacaattgccccacagtgcccaacagcgcccagcaggactcagacagcgatggtcagggtgacgcctgcgacgaggatgacgacaacgacggggtccccgacagtcgggacaactgccgcctggtgcccaacccgggccaggaagacgctgaccgggacggtgtgggcgacgtgtgccagggcgacttcgacgcagacaaggtggtggacaagattgatgtgtgtccggagaacgccgaagtcaccctcaccgacttccgggccttccagacggttgtgttggaccccgagggcgacgcgcaaatagaccccaactgggtggtgctcaaccaggggatggagatcgtgcaaacaatgaacagcgaccctggcctggctgtgggttacacggccttcaatggcgtggacttcgaaggcacgttccacgtgaatacggtcacagatgacgactacgcgggcttcatctttggctaccaggacagctctagcttctacgtggtcatgtggaagcagatggagcagacgtattggcaggcgaaccccttccgagccgtagccgagcccggcatccagctgaaggccgtgaagtcctccacaggccctggggagcagctgcggaatgcactgtggcacacgggggacacagcatcacaggtgcggctgctatggaaggacccccgcaacgtgggctggaaggacaagacatcctaccgctggttcctacaacaccggccccaagtgggctacatcagagtgcggttctatgagggccctgagctggtggccgacagcaacgtggtcttggacacgaccatgcggggcggccgcctaggagtcttctgcttctcccaggagaacatcatctgggccaacctgcgctaccgctgcaatgacaccatccccgaggactacgagatccagcggttgctgcaggcctag

SEQ ID NO: 14 - protein, 755 aa, Equus caballus
MVLSAAPVLLLALAALVSSQGQTPLGTELGPQMLRELQETNAALQDVRELLRQQVKEITFLKNTVMECDACGMQPARTPRVSVRPLAQCAPGSCFPGVACTQTASGARCGPCPAGFTGNGPYCADVNECNANPCFPRVRCINTSPGFRCEACPPGYSGPTHEGVGMAFAKANKQVCTDIDECETGQHNCVPNSVCINTQGSFQCGPCQPGFVGDQASGCRPRAQRFCPDGTPSPCHEKADCVLERDGSRSCVCAVGWAGNGLLCGRDTDLDGFPDEKLRCSERQCRKDNCVTVPNSGQEDADRDGIGDACDTDADGDGVPNEGDNCPLVRNPDQRNTDGDKWGDACDNCRSQKNDDQKDTDQDGRGDACDDDIDGDRIRNAVDNCPRVPNSDQKDSDGDGIGDVCDNCPQKSNPDQRDVDHDFVGDACDSDQDKDGDGHQDSRDNCPTVPNSAQQDSDSDGQGDACDEDDDNDGVPDSRDNCRLVPNPGQEDADRDGVGDVCQGDFDADKVVDKIDVCPENAEVTLTDFRAFQTVVLDPEGDAQIDPNWVVLNQGMEIVQTMNSDPGLAVGYTAFNGVDFEGTFHVNTVTDDDYAGFIFGYQDSSSFYVVMWKQMEQTYWQANPFRAVAEPGIQLKAVKSSTGPGEQLRNALWHTGDTASQVRLLWKDPRNVGWKDKTSYRWFLQHRPQVGYIRVRFYEGPELVADSNVVLDTTMRGGRLGVFCFSQENIIWANLRYRCNDTIPEDYEIQRLLQA

SEQ ID NO: 15 - DNA, 309 bp, Equus caballus
cgtgggagacgcttgtgacagcgaccaagacaaggatggggatgggcaccaggactctcgggacaattgccccacagtgcccaacagcgcccagcaggactcagacagcgatggtcagggtgacgcctgcgacgaggatgacgacaacgacggggtccccgacagtcgggacaactgccgcctggtgcccaacccgggccaggaagacgctgaccgggacggtgtgggcgacgtgtgccagggcgacttcgacgcagacaaggtggtggacaagattgatgtgtgtccggagaacgccgaagtcaccct

SEQ ID NO: 16 - protein, 103 aa, Equus caballus
VGDACDSDQDKDGDGHQDSRDNCPTVPNSAQQDSDSDGQGDACDEDDDNDGVPDSRDNCRLVPNPGQEDADRDGVGDVCQGDFDADKVVDKIDVCPENAEVTL

SEQ ID NO: 17 - DNA, 329 bp, Sus scrofa
cttcaatggcgtggacttcgaaggcacattccacgtgaacacagtcacggatgacgactacgcgggtttcatctttggctaccaagacagttccagcttctatgtggtcatgtggaagcagatggagcagacatactggcaggcaaaccccttccgcgccgtggcggagcctggcatccagctcaaggccgtgaagtcctccacaggccctggggagcagcttcgaaacgccctgtggcacacaggggacacagcatcacaggtgcggctgctgtggaaggacccccgcaacgtgggctggaaggacaagaagtcctatcgttggttcc

SEQ ID NO: 18 - protein, 109 aa, Sus scrofa
FNGVDFEGTFHVNTVTDDDYAGFIFGYQDSSSFYVVMWKQMEQTYWQANPFRAVAEPGIQLKAVKSSTGPGEQLRNALWHTGDTASQVRLLWKDPRNVGWKDKKSYRWF

SEQ ID NO: 19 - DNA, 278 bp, Bovine
  modified_base (32)..(99): N = A, C, G or T/U
gcagaaatgcaagctgggatgccgagggaaanaaggaanatcttctggaagganggaaagnnnnnnnnnnnnnnnnnnnnnnnnnnnnnnnnnnnnnnnagtctctaggaggctgggactgggcacgaatacttggtttaactttgtagttattgggagccaccaaaggtggagtggggactctgtcccagactaatcccaggtctgcacctgctctgctgaagtcagcctaaccccggccccatctggggatccggttctgttcccctgcttctcac

SEQ ID NO: 20 - protein, 6 aa, Artificial Sequence
  Description of Artificial Sequence  Synthetic Peptide
HIDIDD

SEQ ID NO: 21 - DNA, 17 bp, Artificial Sequence
  Description of Artificial Sequence  Synthetic Primer
ataucgauaucgaugau

SEQ ID NO: 22 - DNA, 18 bp, Artificial Sequence
  Description of Artificial Sequence  Synthetic Primer
agugaucgaugcatuacu

SEQ ID NO: 23 - DNA, 21 bp, Artificial Sequence
  Description of Artificial Sequence  Synthetic Primer
gatcatatcgatatcgatgat

SEQ ID NO: 24 - DNA, 22 bp, Artificial Sequence
  Description of Artificial Sequence  Synthetic Primer
ccggagtgatcgatgcattact

SEQ ID NO: 25 - protein, 5 aa, Artificial Sequence
  Description of Artificial Sequence  Synthetic Peptide
GMCSF

SEQ ID NO: 26 - protein, 17 aa, Artificial Sequence
  Description of Artificial Sequence  Synthetic Peptide
GPEDTSRAPENQQKTGC

SEQ ID NO: 27 - protein, 98 aa, Artificial Sequence
  Description of Artificial Sequence  Synthetic Peptide
MPSSVSWGILLLAGLCLVPVSLAEDLNQRGTELRSPSVDLNKPGRHSEPAAAGDLAPQMLRELQETNAALQDVRELLRQQVKEITFLKNTVMECDACG

SEQ ID NO: 28 - protein, 23 aa, Artificial Sequence
  Description of Artificial Sequence  Synthetic Peptide
MPSSVSWGILLLAGLCLVPVSLA

SEQ ID NO: 29 - protein, 20 aa, Artificial Sequence
  Description of Artificial Sequence  Synthetic Peptide
NQRGTELRSPSVDLNKPGRH

SEQ ID NO: 30 - protein, 45 aa, Artificial Sequence
  Description of Artificial Sequence  Synthetic Peptide
DLAPQMLRELQETNAALQDVRELLRQQVKEITFLKNTVMECDACG

SEQ ID NO: 31 - protein, 8 aa, Artificial Sequence
  Description of Artificial Sequence  Synthetic Peptide
DDHIDIDD

SEQ ID NO: 32 - protein, 23 aa, Artificial Sequence
  Description of Artificial Sequence  Synthetic Peptide
DDLQAVHAAHAEINEADHIDIDD

SEQ ID NO: 33 - protein, 12 aa, Artificial Sequence
  Description of Artificial Sequence  Synthetic Peptide
QAVHAAHAEINE

SEQ ID NO: 34 - protein, 68 aa, Artificial Sequence
  Description of Artificial Sequence  Synthetic Peptide
DDPGGSILMQYIKANSKFIGITELKKLGGSNDIFNNFTVSFWLRVPKVSASHLEQYGGGSGDHIDIDD

SEQ ID NO: 35 - protein, 15 aa, Artificial Sequence
  Description of Artificial Sequence  Synthetic Peptide
QYIKANSKFIGITEL

SEQ ID NO: 36 - protein, 23 aa, Artificial Sequence
  Description of Artificial Sequence  Synthetic Peptide
FNNFTVSFWLRVPKVSASHLEQY

(19) United States
(12) Patent Application Publication
(10) Pub. No.: US 2005/0137156 A1
(43) Pub. Date: Jun. 23, 2005

(54) Methods and compositions for generating an immune response

(76) Inventors: Stephen Albert Johnston, Dallas, TX (US); Ross S. Chambers, Dallas, TX (US); Kathryn F. Sykes, Dallas, TX (US)

Correspondence Address: FULBRIGHT & JAWORSKI L.L.P., 600 CONGRESS AVE., SUITE 2400, AUSTIN, TX, 78701, US

(21) Appl. No.: 10/914,714

(22) Filed: Aug. 9, 2004

Related U.S. Application Data
(60) Provisional application No. 60/493,524, filed on Aug. 9, 2003.

(51) Int. Cl. A61K 48/00; C12Q 1/68; C07H 21/04

(52) U.S. Cl. 514/44; 435/6; 536/23.2

(57) ABSTRACT

The present invention provides a method for enhancing an immune response in a subject by providing to a subject a genetic immunization vector comprising a nucleic acid sequence encoding a COMP domain linked to a an antigen domain.

This application claims the benefit of U.S. Provisional Application Ser. No. 60/493,542 filed Aug. 9, 2003, the entire contents and disclosure of which are specifically incorporated by reference herein without disclaimer. The government owns rights in the present invention pursuant to grants from the Programs for Genomic Applications from the U.S. National Heart, Lung and Blood Institute, number U01HL66880.

### BACKGROUND OF THE INVENTION

1. Field of the Invention

The present invention relates generally to the fields of immunopreventive therapy and vaccine development. More particularly, it concerns polypeptides and nucleic acids encoding such polypeptides that can be used to initiate, stimulate, and/or enhance an immune response. These polypeptides and nucleic acids encoding them can be used as adjuvants that can be used to generate more potent and robust immunological responses against desired polypeptides.

2. Description of Related Art

Many methodologies of medical treatment can be envisioned that will require or benefit from an ability to initiate, stimulate, and/or enhance an immune response in the context of genetic immunization. These methodologies include those depending upon the creation of an immune response against a desired antigenic polypeptide and those that depend upon the initiation or modulation of an innate immune response.

Whole genome sequencing has led to the discovery of tens of thousands of putative genes. The rate of genome sequencing far exceeds the ability to match it with an understanding of the encoded proteome. Antibodies are key tools in leading quantitative investigations of the encoded proteins, but current methods for producing antibodies have become a rate-limiting step (Kodadek, 2001). A major drawback in most methods for generating antibodies or antibody-like molecules, is the requirement for at least microgram quantities of purified protein. Purification of proteins is laborious and, moreover, can be difficult if a particular protein cannot be overexpressed. A general solution to this problem is to develop genetic-based methods for isolating antibodies.

Technology for producing antibodies based on genetic immunization has been developed (Tang et al., 1992). Genetic immunization-based antibody production offers numerous advantages including; high throughput since the DNA constructs can be rapidly produced (Sykes and Johnston, 1999), high specificity since the immunizing material is pure DNA, and antibodies produced from genetically immunized animals are more likely to recognize the native protein (Tang et al., 1992). Nonetheless, genetic immunization has received relatively little attention as a method for producing antibodies for proteomic applications. One reason for this, has been the variable success of genetic immunization in producing antibodies (Babiuk et al., 1999).

The use of adjuvants for immunization are well known in the art; however, the challenge of developing safe and effective adjuvants is ongoing. A primary disadvantage with current adjuvants is that most are unsuitable for use in human vaccines, especially genetic vaccines.

One of the first adjuvants developed was Freund's complete adjuvant. This adjuvant has excellent immunopotentiating properties, however, its side effects are so severe that it renders the use of this adjuvant unacceptable in humans, and sometimes in animals. Other oil emulsions adjuvants such as Incomplete Freund's Adjuvant (IFA); Montanide ISA (incomplete seppic adjuvant); Ribi Adjuvant System (RAS); TiterMax; and Syntex Adjuvant Formulation (SAF) are also associated with various side effects such as toxicity and inflammation. Oil based adjuvants in general are less desirable in genetic immunization; they create side effects such as visceral adhesions and melanized granuloma formations, and they cannot form a homogeneous mixture with DNA preparations such as DNA-based vaccines.

Bacterially derived adjuvants, such as MDP and lipid A are also associated with undesirable side effects. Bacterial products such as Bordetella pertussis, Corynebacterium granulosum derived P40 component, lipopolysaccharide (LPS), Mycobacterium and its components, and Cholera toxin, are another preferred group of adjuvants. However, although they may augment the immune response to other antigens they are associated with side effects, such as epilepsy as in the case of B. pertussis, and varying levels of toxicity.

Mineral compounds which include aluminum phosphate or aluminum hydroxide (alum) and calcium phosphate as adjuvants may also be employed. Aluminum salt-based adjuvants (such as alum) have excellent safety records but poor efficacy with some antigens (Sjolander et al., 1998). They are the most frequently used adjuvants for vaccine antigen delivery presently. Aluminum salt-based adjuvants are generally weaker adjuvants than emulsion adjuvants. The most widely used is the antigen solution mixed form with pre-formed aluminum phosphate or aluminum hydroxide; however, these vaccines are difficult to manufacture in a physico-chemically reproducible way, which results in batch to batch variation of the vaccine. When used in large quantity, an inflammatory reaction may occur at the site of injection that is generally resolved in a few weeks although chronic granulomas may occasionally form.

Other available adjuvants are known to those skilled in the art. One such adjuvant includes liposomes. Although liposomes show favorable characteristics for use in bulk vaccine preparations, the preparation proves to be rather complex for use with occasional antigens prepared for injection, especially when the antigen is available in limited quantity. Gerbu^{R} adjuvant is an aqueous phase adjuvant that is associated with minimal inflammatory effects, but may require frequent boosting to maintain high titer. Squalene, also included in the group of adjuvants, has been associated with the Gulf War Syndrome and includes such side effects as arthritis, fibromayalgia, rashes, chronic headaches, sclerosis and non healing skin lesions to name a few.

Various polysaccharide adjuvants are also known to those skilled in the art. For example, Yin et al. (1989) describe the use of various pneumococcal polysaccharide adjuvants on the antibody responses of mice. The doses that produce optimal responses, or that otherwise do not produce suppression, as indicated in Yin et al. (1989), should be employed. Polyamine varieties of polysaccharides are particularly preferred, such as chitin and chitosan, including deacetylated chitin. Hence, more effective adjuvants are needed that will enhance the immune response induced by genetic vaccines.

### SUMMARY OF THE INVENTION

The present invention overcomes the deficiencies in the art by identifying polypeptides that are useful in modulating immune responses to antigens and nucleic acid sequences that encode such polypeptides. For example, the applicants have identified COMP sequences that can be employed in these manners.

In some general embodiments, the invention relates to methods of initiating or enhancing an immune response in a subject comprising administering to the subject a nucleic acid comprising a sequence encoding a COMP domain and a nucleic acid comprising a sequence encoding an antigen domain. In some preferred embodiments, the sequence encoding the COMP domain and the sequence encoding an antigen domain are comprised in the same nucleic acid. In many embodiments the nucleic acid has the COMP domain functionally linked to the antigen domain.

In preferred embodiments of the invention, the nucleic acids of the invention are expressed in the subject as a fusion protein comprising a COMP domain and an antigen domain. However, in other embodiments the COMP domain and the antigen domain may be expressed as separate peptides within the subject. In such embodiments, the COMP domain serves as an adjuvant to initiate or enhance an immune response to the antigen. This immune response can then be directed against a disease and/or serve to protect the subject against disease. For example, the immune response can protect the subject against pathogenic infection, viral infection, cancer/malignancy, and/or any other disease state that is preventable or treatable by vaccination. In this regard, the invention relates to methods of genetic immunization and/or vaccination, in which an antibody or antibodies against the antigen are produced in the subject.

The nucleic acids of the present invention may be introduced into a subject in any manner effective to bring about the desired results. For example, the nucleic acids may be introduced by inhalation, by gene gun, or by injection into the subject.

In preferred embodiments of the invention, the subject is a mammal or a bird. For example, the subject may be a human, rat, mouse, cow, pig, horse, or chicken. Immunization may be performed for several reasons. First, one may wish to vaccinate a human or animal subject, such as an agricultural animal, to protect the subject against disease. Also, one may wish to immunize an animal to be a source of antibodies against the antigen. In this regard, the use of a bird system has some advantages, because, in order to harvest antibodies, it is merely necessary to break open a bird egg, rather than to kill, or at least bleed, the animal.

In most embodiments, the nucleic acid is further defined as a vector, and can be produced according to any of the methods known to those of skill in the art and/or disclosed herein. Such a vector may contain the COMP domain encoding nucleic acid and the antigen encoding nucleic acid in cis or in trans. Further, within the vector, the COMP domain encoding region and the antigen encoding region may be in any order. Further, the vector may comprise sequences encoding multiple COMP domains and/or antigen domains. For example, it is understood that some embodiments of the invention may beneficially comprise at least two, three, or more COMP domains, which may be identical or different. These vectors may comprise nucleic acid domains of any of a number of additional elements, including promoters, enhancers, targeting peptide encoding domains, secretory peptide encoding domains, etc. In some embodiments, the vector comprises certain chemically synthesized promoters described in U.S. application Ser. No. 10/781,055, entitled “RATIONALLY DESIGNED AND CHEMICALLY SYNTHESIZED PROMOTER FOR GENETIC VACCINE AND GENE THERAPY,”by Johnston et al., filed Feb. 18, 2003, the entire contents and disclosure of which relating to specific promoters and any relevant techniques are hereby incorporated by reference herein for all purposes. In some embodiments, the vector comprises a secretory leader sequence linked to the nucleic acid sequence comprising a COMP domain by a non-immunogenic peptide sequence. In such cases, the non-immunogenic peptide can be a cell-targeting peptide, for example, a dendritic cell-targeting peptide. Of course, the invention relates to all of the above-described vectors specifically, both independently and in the context of methods disclosed herein.

In certain embodiments of the present invention, a COMP polypeptide may be administered with an antigen to a subject to intitate, stimulate, and/or promote an immune response. Preferably, in these embodiments, multiple COMP polypeptides are administered in a pharmaceutically acceptable carrier. The multiple COMP polypeptides may be the same COMP polypeptide or different COMP polypeptides. The COMP polypeptide may be a naturally-occurring COMP polypeptide, or it may be mutated or truncated as compared to a naturally-occurring COMP polypeptide. The subject may be a mammal or a bird, and in some embodiments use of a bird system may be preferable.

The COMP domains useful in the context of the invention may be any of the variety of COMP domains that may be determined to have the adjuvant activity disclosed in the current specification. One of skill may employ any of the techniques taught herein and/or known to those of skill in order to prepare, test, and employ such sequences. For example, all or part of any of the amino acid sequences of the specific COMP proteins set forth in SEQ ID NO:2; SEQ ID NO:5; SEQ ID NO:7; SEQ ID NO:10; SEQ ID NO:12; SEQ ID NO:14; SEQ ID NO:16 and SEQ ID NO:18, will be useful in the context of the present invention. Of course, the invention is in no way limited to the use of COMP domains that are from these specific sequences. Rather, those of skill understand that there may be other currently known, or later discovered, COMP proteins that can be used as the basis of COMP domains for use in the invention. For example, one of skill will be able to use information relating to these specific COMP proteins to search any of the various amino acid and/or nucleic acid sequence databases for homologues and related proteins that will contain COMP domains for use in the present invention. Further, those of skill will be able to use known molecular biology procedures, in combination with currently known or later learned sequence information relating to COMP, to characterize related proteins and obtain COMP domains that may be used in the context of the invention. Further, using methods disclosed herein and/or known to those of skill, one will be able to mutate or modify naturally occurring COMP domains to obtain COMP domain variants for use in the context of the application. In some preferred embodiments, the COMP domains employed in the invention will be less than full-length segments of any given COMP protein. For example, the COMP domain may comprise or consist of at least 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 55, 60, 65, 70, 80, 90, 100, 125, 150, 175, 200, 225, 250, 300, 350, 400, 450, 500, 600, 700, 800, and/or any other integer between 5 and the number of amino acids in the given COMP protein, contiguous amino acids of the amino acid sequence of any full-length COMP polypeptide. In some preferred embodiments, the COMP domain has the sequence of SEQ ID NO:30. Further, the COMP domains of the invention may be at least 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 55, 60, 65, 70, 80, 90, 100, 125, 150, 175, 200, 225, 250, 300, 350, 400, 450, 500, 600, 700, 800, and/or any other integer between 5 and the number of amino acids in the given COMP protein, amino acids in length.

Nucleic acid sequences encoding the COMP domains of the present invention may be prepared or obtained in any method known to those of skill in the art. For example, in some embodiments, the nucleic acid sequence encoding a given COMP domain will be a native nucleic acid sequence that has all or part of genetic sequence encoding the COMP domain. Alternatively, the nucleic acid sequences may be modified relative to a native nucleic acid, via either methods of genetic sequence manipulation or synthesis. Modified nucleic acids may encode a native COMP domain amino acid sequence, or may encode a variant or mutant of such a sequence. Some nucleic acid sequences for use in the present invention will comprise or consist of all or part of the nucleic acid sequences in any of SEQ ID NO:1; SEQ ID NO:3; SEQ ID NO:4; SEQ ID NO:6; SEQ ID NO:8; SEQ ID NO:9; SEQ ID NO:11; SEQ ID NO:13; SEQ ID NO:15; SEQ ID NO:18 and SEQ ID NO:19. For example, a COMP domain may be encoded by a nucleic acid comprising or consisting of at least 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 55, 60, 65, 70, 80, 90, 100, 125, 138, 150, 175, 200, 225, 250, 300, 350, 400, 450, 500, 600, 700, 800, 900, 1,000, 1,250, 1,500, 1,750, 2,000, 2,250, and/or any other integer between 15 and the number of nucleic acids encoding a given COMP protein, contiguous nucleic acids of a nucleic acid sequence of any full-length COMP polypeptide. Further, the COMP domains of the invention may be encoded by a nucleic acid of at least 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 55, 60, 65, 70, 80, 90, 100, 125, 138, 150, 175, 200, 225, 250, 300, 350, 400, 450, 500, 600, 700, 800, 900, 1,000, 1,250, 1,500, 1,750, 2,000, 2,250, and/or any other integer between 15 and the number of nucleic acids encoding a given COMP protein, in length.

The antigen domains of the present invention may be any polypeptide sequence against which any form of immune response is desired. Those of ordinary skill will be able to follow the teachings of the specification and/or use their knowledge to determine such sequences. In some embodiments, one of skill might determine antigen using the methodologies disclosed in U.S. Pat. No. 5,989,553, entitled “Expression Library Immunization” and/or in U.S. Pat. No. 6,410,241, entitled “Methods of screening open reading frames to determine whether they encode polypeptides with an ability to generate an immune response,” the entire contents and disclosures of which relating to any and all relevant techniques are hereby incorporated by reference herein for all purposes.

In conformance with long-standing patent law, the use of the articles “a” and “an” in combination with the conjunction “comprising” mean “one or more than one” and “at least one.”

### BRIEF DESCRIPTION OF THE DRAWINGS

The following drawings form part of the present specification and are included to further demonstrate certain aspects of the present invention. The invention may be better understood by reference to one or more of these drawings in combination with the detailed description of specific embodiments presented herein.

FIG. 1. Genetic immunization vector design. The plasmids pBQAP10, pBQAP-OVA, and pBQAP-TT all contained the SP72 promoter and the rabbit β-globin terminator flanking the expression cassette shown above. The pCMVi10 plasmid is identical to pBQAP10 except it contains the CMV promoter. The sequence HIDIDD (SEQ ID NO:20) is encoded by the 5′ flanks included in the PCR™ primers used to amplify the antigen gene.

FIGS. 2A-2C. Antibody responses of mice immunized with pBQAP10-AAT. Groups of five Balb/C mice were either immunized with pBQAP10-AAT alone (squares), with a GM-CSF plasmid (triangles), or with both GM-CSF and Flt3L plasmids (circles; FIG. 2A). Antibodies against AAT were measured using ELISA and converted to monoclonal antibody equivalents using an anti-AAT monoclonal antibody of known concentration. The slopes of the curves for dilutions of the sera and the monoclonal antibody were similar. Sera were diluted 1:250,1:250, 1:1000 and 1:6000 for the zero, two, five and seven week samples respectively. Arrows indicate immunizations and bars indicate standard errors. FIG. 2B—Individual antibody levels measured by ELISA for the group of five mice immunized three times with the AAT, GMCSF and Flt3L plasmids and a group of five mice immunized once with AAT protein. FIG. 2C—Western blot analysis of sera pooled from 5 mice immunized as described in A. Control lane contains 10 μg of a whole cell extract from E. coli with 50 ng of a GST fusion protein unrelated to AAT. The AAT and tag lanes are the same as the control lane except with 50 ng of pure AAT, and 50 ng of GST-tag, respectively. Sera were diluted 1:5000.

FIG. 3. Western blot analysis of antibodies generated using genetic immunization. Each lane contains 10 μg of an E. coli whole cell extract with either 50 ng of an unrelated GST fusion protein (lane 1), or the GST antigen (lane 2). Sera from mice was diluted 1:5000 and used to probe the blots.

FIG. 4. Western blot analysis of natural extracts. All antibodies were diluted 1:1000. The antibodies raised against the Mtb proteins were used to probe western blots containing 3.25 μg of a Mycobacterium tuberculosis whole cell extract (Mtb. ext.). As a control the antibodies were used to probe a western blot containing 10 μg of an E. coli whole cell extract with either 50 ng of an unrelated GST fusion protein (control), or the relevant GST antigen. The TAF250 antibody was probed against 4.5 μg of a HeLa nuclear extract (HNE) or 6 μg of a yeast extract (YE). The AAT, ApoAI and ApoD antibodies were probed against 7 μg of human sera or as a control 25 μg of a human brain extract. The myoglobin, FABP, TrC, and TrI antibodies were probed against 25 μg of either human brain, liver or heart extract. Arrows indicate the known sizes of the mature proteins.

FIG. 5. Sensitivity of antibodies. Each lane contains 10 μg of an E. coli whole cell extract with either 0.5, 5, or 50 ng of the GST antigen. Sera from mice was diluted 1:5000 and used to probe the blots.

FIGS. 6A-6B. Antigen Structure (FIG. 6A). Genetic Immunization Vectors Containing COMP (FIG. 6B).

FIG. 7. Testing of hAAT Antibodies by ELISA.

FIG. 8. COMP Increases Specific Antibody Levels.

FIG. 9. Anti-AAT Antibody Levels Post-Immunization.

FIG. 10. Generation of Significant Antibody titers Using a COMP linked in cis to a Antigen.

FIG. 11. COMP Causes an Elevated Humoral Response.

FIG. 12. Vectors constructs: RAN-COMP-TT-Ag, Ag-linker-COMP-TT, COMP-TT-Ag.

FIG. 13. Measurement Of Antibody Titers Following A Boost.

FIG. 14. Antibody production in Chicken.

### DESCRIPTION OF ILLUSTRATIVE EMBODIMENTS

The primary purpose of an adjuvant is to enhance the immune response to a particular antigen of interest. There are only few effective adjuvants available today and only one approved for the clinics. The Cartilage Oligomeric Matrix Protein (COMP) not only provides an alternative but also holds a number of advantages over other adjuvants. COMP is: 1) effectively combined with GI, but is not likely to be restricted to GI; 2) has the potential to be as, or even more effective than known cytokine adjuvants; 3) non-toxic; 4) effective without a large carrier molecule; 4) less expensive and simpler to produce than alternatives, and 5) non-immunogenic.

Thus, the present invention provide COMP as an adjuvant to enhance the immune responses to genetic and protein antigens. COMP is a pentameric glycoprotein of the thrombospondin family that is synthesized by cartilage and tendon. Its small oligomerizing domain is positioned at the N-terminus of the protein. Previous studies have shown that fusion of this domain to another protein can lead to chimeric pentamers inside the cell.

The COMP technology presented in this invention provides a solution to the low levels of immune reactivity of genetic immunization. COMP is an effective GI adjuvant, especially for antibody production. It is non-toxic and endogenous, eliminating problems associated with undesirable side-effects or immune responses raised against an adjuvant carrier. To date, some GI protocols have included a cytokine to bolster reactivity. This has led to encouraging but, in some cases, hard to uncontrol effects on immunity. COMP is not a cytokine and, therefore, its in vivo effects might be more controlled. This invention would significantly reduce the amount of genetic material needed to elicit a potent and specific immune response in a host animal, thereby reducing production time and costs, while increasing safety. The present invention provides vaccines that are more effective, safer, and cheaper. Large-scale vaccination programs would be more flexible and feasible.

The present invention distinguishes from that of the art (e.g., Hensley et al., 2000, WO 00/01801 and Terskikh et. al., 1998, WO 98/18943) in that it provides a system that can enhance immunogenecity of any protein fused to COMP. Additionally, COMP provides several advantages over the FtsZ vaccine (WO 00/01801) in that COMP is a small molecule of (46 amino acids versus that of FtsZ (390 amino acids). The COMP plasmid of the present invention encodes a scaffold that is fused to antigens to enhance antibody responses. The scaffold includes several components that assist in producing antibody reagents to proteins. These include a secretion leader sequence, an antigenic tag as an internal control, COMP to enhance solubility, secretion and by multimerizing enhance antigen uptake and presentation, and T cell epitopes to ensure T cell help. Together they comprise a robust system that is demonstrated to efficiently raise antibodies to a wide range of antigens, including antigens that are known to be poorly immunogenic. In addition, COMP is not immunogenic which is further advantageous in that makes antibodies to antigens and eliminates other components that may interfere with the diagnostic or therapeutic application. Moreover, COMP is provided in the present invention for use in genetic immunization and comprises of additional nucleic acid sequences encoding a leader sequence and antigenic tag which is distinguished from that in the art (WO 98/18943).

### I. THE PRESENT INVENTION

In the present invention, enhancement of an immune response is mediated by a nucleic acid encoding a COMP domain which increases the humoral response to an antigen. The present invention provides a method of such enhancement of an immune response in a mammalian subject such as a human, pig, horse, cow, rat or mouse, by contacting the subject with a nucleic acid encoding a COMP domain linked to a portion encoding an antigen domain.

The present invention demonstrates that the pentamerizing domain of the COMP gene is a naturally occurring molecular coupler that confers adjuvant-like activity without toxicity. Genetic fusion of the COMP oligomerization domain to the N-terminus of antigens achieved immune enhancement without the untoward side effects inherent to carrier molecules and chemical adjuvants. The COMP fusion antigens were delivered as bacterially propagated plasmids or as synthetically built linear expression elements. The small size of the COMP domain (50 amino acids) proved ideal for the synthetic applications. The adjuvant effect of COMP was observed on fused antigens, indicating that particular components of a mixed vaccine innoculum might be designated for modulation without influencing other components.

In the present invention it is shown that a genetic immunization-based system can be used to efficiently raise useful antibodies against a wide range of antigens. This system has been tested by immunizing mice with more than 130 antigens and have demonstrated a final success rate of 84%.

Following genetic immunization (GI), in mice, with the COMP fused to antigen construct, a 2 to 10-fold increase in antigen-specific antibody reactivities was observed as compared to mice from GI with the same expression vector minus the COMP sequences. A number of different types of antigens have been tested, such as viral, cytoplasmic HIV gag and human, secreted alpha anti-trypsin (AAT). COMP was shown to perform better as an adjuvant than the widely-used cytokine gene GMCSF. Likewise, a COMP-fused antigen construct conferred better host survival than the same construct without COMP in a viral-challenge assay.

### II. NUCLEIC ACIDS ENCODING COMP POLYPEPTIDES

The present invention identifies nucleic acids encoding peptides that enhance an immune response to an antigen. More specifically, the present invention identifies nucleic acid sequences encoding a COMP domain, that have such activity. SEQ ID NO:1; SEQ ID NO:3; SEQ ID NO:4; SEQ ID NO:6; SEQ ID NO:8; SEQ ID NO:9; SEQ ID NO:11; SEQ ID NO:13; SEQ ID NO:15; SEQ ID NO:18 and SEQ ID NO:19 are the COMP sequences that are contemplated in the present invention, with the respective amino acid sequences provided in SEQ ID NO:2; SEQ ID NO:5; SEQ ID NO:7; SEQ ID NO:10; SEQ ID NO:12; SEQ ID NO:14; SEQ ID NO:16 and SEQ ID NO:18. Accordingly, in certain exemplary aspects, the present invention concerns nucleic acid sequences that encode proteins, polypeptides or peptides that express adjuvant activity.

The nucleic acid may be derived from genomic DNA, i.e., cloned directly from the genome of a particular organism. Alternatively, the nucleic acid sequence can ge synthetically built. In the case of synthetic nucleic acids, one can determine a series of codons that encode COMP and also are selected for optimal performance in a target organism. A nucleic acid generally refers to at least one molecule or strand of DNA, RNA or a derivative or mimic thereof, comprising at least one nucleobase, such as, for example, a naturally occurring purine or pyrimidine base found in DNA (e.g., adenine “A,” guanine “G,” thymine “T,” and cytosine “C”) or RNA (e.g. A, G, uracil “U,” and C). The term nucleic acid encompasses the terms oligonucleotide and polynucleotide. The term oligonucleotide refers to at least one molecule of between about 3 and about 100 nucleobases in length. The term polynucleotide refers to at least one molecule of greater than about 100 nucleobases in length. These definitions generally refer to at least one single-stranded molecule, but in specific embodiments will also encompass at least one additional strand that is partially, substantially or fully complementary to the at least one single-stranded molecule. Thus, a nucleic acid may encompass at least one double-stranded molecule or at least one triple-stranded molecule that comprises one or more complementary strand(s) or “complement(s)” of a particular sequence comprising a strand of the molecule.

As used in this application, the term a nucleic acid encoding a COMP domain, refers to a nucleic acid molecule that has been isolated free of total genomic nucleic acid. In preferred embodiments, the invention concerns a nucleic acid sequence essentially as set forth in SEQ ID NO:1; SEQ ID NO:3; SEQ ID NO:4; SEQ ID NO:6; SEQ ID NO:8; SEQ ID NO:9; SEQ ID NO:11; SEQ ID NO:13; SEQ ID NO:15; SEQ ID NO:18 or SEQ ID NO:19. The term as set forth in SEQ ID NO:1; SEQ ID NO:3; SEQ ID NO:4; SEQ ID NO:6; SEQ ID NO:8; SEQ ID NO:9; SEQ ID NO:11; SEQ ID NO:13; SEQ ID NO:15; SEQ ID NO:18 or SEQ ID NO:19 means that the nucleic acid sequence substantially corresponds to a portion or all of SEQ ID NO:1; SEQ ID NO:3; SEQ ID NO:4; SEQ ID NO:6; SEQ ID NO:8; SEQ ID NO:9; SEQ ID NO:11; SEQ ID NO:13; SEQ ID NO:15; SEQ ID NO:18 and SEQ ID NO:19.

It also is contemplated that a given nucleic acid sequence such as a COMP sequence may be represented by natural variants that have slightly different nucleic acid sequences but, nonetheless, encode the same protein (Table 1). Furthermore, the term functionally equivalent codon is used herein to refer to codons that encode the same amino acid, such as the six codons for arginine or serine (Table 1), and also refers to codons that encode biologically equivalent amino acids, as discussed herein. As discussed elsewhere in the specification, one can synthetically create codon-optimized COMP encoding nucleic acids that will have improved and/or maximal expression in a desired host.

As used herein, the term DNA segment refers to a DNA molecule that has been isolated free of total genomic DNA of a particular species. Therefore, a DNA segment encoding a polypeptide refers to a DNA segment that contains the polypeptide-coding sequences yet is isolated away from, total genomic DNA. Included within the term “DNA segment” are a polypeptide or polypeptides, DNA segments smaller than a polypeptide, and recombinant vectors, including, for example, plasmids, cosmids, phage, viruses, and the like.

A DNA segment comprising an isolated COMP domain refers to a DNA segment including COMP domain or other similar gene coding sequences and, in certain aspects, regulatory sequences, isolated substantially away from other naturally occurring genes or protein encoding sequences. In this respect, the term gene is used for simplicity to refer to a functional protein, polypeptide or peptide encoding unit. As will be understood by those in the art, this functional term includes both genomic sequences, cDNA sequences and smaller or bigger engineered gene segments that express, or may be adapted to express, proteins, polypeptides or peptides.

In other embodiments, the invention concerns isolated DNA segments and recombinant vectors incorporating DNA sequences that encode a polypeptide or peptide that includes within its amino acid sequence a contiguous amino acid sequence in accordance with, or essentially corresponding to the polypeptide.

It is contemplated that the nucleic acid constructs of the present invention may encode full-length polypeptide from any source. A nucleic acid sequence may encode a full-length polypeptide sequence with additional heterologous coding sequences, for example to allow for purification of the polypeptide, transport, secretion, post-translational modification, or for therapeutic benefits such as targeting or efficacy. A tag or other heterologous polypeptide may be added to the modified polypeptide-encoding sequence, wherein heterologous refers to a polypeptide that is not the same as the modified polypeptide.

In a non-limiting example, one or more nucleic acid constructs may be prepared that include a contiguous stretch of nucleotides identical to or complementary to the a particular gene, such as the COMP genes SEQ ID NO:1; SEQ ID NO:3; SEQ ID NO:4; SEQ ID NO:6; SEQ ID NO:8; SEQ ID NO:9; SEQ ID NO:11; SEQ ID NO:13; SEQ ID NO:15; SEQ ID NO:18 and SEQ ID NO:19. A nucleic acid construct may be at least 20, 30, 40, 50, 60, 70, 80, 90, 100, 110, 120, 130, 140, 150, 160, 170, 180, 190, 200, 250, 300, 400, 500, 600, 700, 800, 900, 1,000, 2,000, 3,000, 4,000, 5,000, 6,000, 7,000, 8,000, 9,000, 10,000, 15,000, 20,000, 30,000, 50,000, 100,000, 250,000, 500,000, 750,000, to at least 1,000,000 nucleotides in length, as well as constructs of greater size, up to and including chromosomal sizes (including all intermediate lengths and intermediate ranges), given the advent of nucleic acids constructs such as a yeast artificial chromosome are known to those of ordinary skill in the art. It will be readily understood that intermediate lengths and intermediate ranges, as used herein, means any length or range including or between the quoted values (i.e., all integers including and between such values).

The DNA segments used in the present invention encompass biologically functional equivalent modified polypeptides and peptides. Such sequences may arise as a consequence of codon redundancy and functional equivalency that are known to occur naturally within nucleic acid sequences and the proteins thus encoded. Alternatively, functionally equivalent proteins or peptides may be created via the application of recombinant DNA technology, in which changes in the protein structure may be engineered, based on considerations of the properties of the amino acids being exchanged. Changes designed by human may be introduced through the application of site-directed mutagenesis techniques, e.g., to introduce improvements to the antigenicity of the protein, to reduce toxicity effects of the protein in vivo to a subject given the protein, or to increase the efficacy of any treatment involving the protein.

In addition to their use in generating an immune response, the nucleic acid sequences contemplated herein also have a variety of other uses. For example, they also have utility as probes or primers in nucleic acid hybridization embodiments. As such, it is contemplated that nucleic acid segments that comprise a sequence region that consists of at least a 14 nucleotide long contiguous sequence that has the same sequence as, or is complementary to, a 14 nucleotide long contiguous DNA segment will find particular utility. Longer contiguous identical or complementary sequences, e.g., those of about 20, 30, 40, 50, 100, 200, 500, 1000 (including all intermediate lengths) and even up to full length sequences will also be of use in certain embodiments.

The ability of such nucleic acid probes to specifically hybridize to peptide-encoding sequences will enable them to be of use in detecting the presence of complementary sequences in a given sample. However, other uses are envisioned, including the use of the sequence information for the preparation of mutant species primers, or primers for use in preparing other genetic constructions.

TABLE 1

| | |
|---|---|
| Amino Acids | Codons |

| | | | |
|---|---|---|---|
| Alanine | Ala | A | GCA GCC GCG GCU |
| Cysteine | Cys | C | UGC UGU |
| Aspartic acid | Asp | D | GAC GAU |
| Glutamic acid | Glu | E | GAA GAG |
| Phenylalanine | Phe | F | UUC UUU |
| Glycine | Gly | G | GGA GGC GGG GGU |
| Histidine | His | H | CAC CAU |
| Isoleucine | Ile | I | AUA AUC AUU |
| Lysine | Lys | K | AAA AAG |
| Leucine | Leu | L | UUA UUG CUA CUC CUG CUU |
| Methionine | Met | M | AUG |
| Asparagine | Asn | N | AAC AAU |
| Proline | Pro | P | CCA CCC CCG CCU |
| Giutamine | Gln | Q | CAA CAG |
| Arginine | Arg | R | AGA AGG CGA CGC CGG CGU |
| Serine | Ser | S | AGC AGU UCA UCC UCG UCU |
| Threonine | Thr | T | ACA ACC ACG ACU |
| Valine | Val | V | GUA GUC GUG GUU |
| Tryptophan | Trp | W | UGG |
| Tyrosine | Tyr | Y | UAC UAU |

Allowing for the degeneracy of the genetic code, sequences that have at least about 50%, usually at least about 60%, more usually about 70%, most usually about 80%, 5 preferably at least about 90% and most preferably about 95% of nucleotides that are identical to the nucleotides of SEQ ID NO:1; SEQ ID NO:3; SEQ ID NO:4; SEQ ID NO:6; SEQ ID NO:8; SEQ ID NO:9; SEQ ID NO:11; SEQ ID NO:13; SEQ ID NO:15; SEQ ID NO:18 and SEQ ID NO:19 will be sequences that are as set forth in SEQ ID SEQ ID NO:1; SEQ ID NO:3; SEQ ID NO:4; SEQ ID NO:6; SEQ ID NO:8; SEQ ID NO:9; SEQ ID NO:11; SEQ ID NO:13; SEQ ID NO:15; SEQ ID NO:18 and SEQ ID NO:19. Sequences that are essentially the same as those set forth in SEQ ID NO:1; SEQ ID NO:3; SEQ ID NO:4; SEQ ID NO:6; SEQ ID NO:8; SEQ ID NO:9; SEQ ID NO:11; SEQ ID NO:13; SEQ ID NO:15; SEQ ID NO:18 and SEQ ID NO:19 also may be functionally defined as sequences that are capable of hybridizing to a nucleic acid segment containing the complement of SEQ ID NO:1; SEQ ID NO:3; SEQ ID NO:4; SEQ ID NO:6; SEQ ID NO:8; SEQ ID NO:9; SEQ ID NO:11; SEQ ID NO:13; SEQ ID NO:15; SEQ ID NO:18 and SEQ ID NO:19 under standard conditions.

The DNA segments of the present invention include those encoding biologically functional equivalent COMP proteins and peptides, as described above. Such sequences may arise as a consequence of codon redundancy and amino acid functional equivalency that are known to occur naturally within nucleic acid sequences and the proteins thus encoded. Alternatively, functionally equivalent proteins or peptides may be created via the application of recombinant DNA technology, in which changes in the protein structure may be engineered, based on considerations of the properties of the amino acids being exchanged. Changes designed by man may be introduced through the application of site-directed mutagenesis techniques, through gene building technologies, or via random generation and screening for desired function, as described herein and understood to those of skill in the art.

It will also be understood that nucleic acid sequences (and their encoded amino acid sequences) may include additional residues, such as additional 5′ or 3′ sequences (or N- or C-terminal amino acids), and yet still be essentially as set forth in one of the sequences disclosed herein, so long as the sequence meets the criteria set forth above, including the maintenance of biological protein activity where protein expression is concerned. The addition of terminal sequences particularly applies to nucleic acid sequences that may, for example, include various non-coding sequences flanking either of the 5′ or 3′ portions of the coding region or may include various internal sequences, i.e., introns, which are known to occur within genes.

Excepting intronic or flanking regions of any related gene, and allowing for the degeneracy of the genetic code, sequences that have between about 70% and about 80%; or more preferably, between about 81% and about 90%; or even more preferably, between about 91% and about 99% of nucleotides that are identical to the nucleotides of a disclosed sequence are thus sequences that are essentially as set forth in the given sequence.

### III. COMP POLYPEPTIDES

Nucleic acids of the present invention further encodes polypeptide adjuvants as provided herein by SEQ ID NO:2; SEQ ID NO:5; SEQ ID NO:7; SEQ ID NO:10; SEQ ID NO:12; SEQ ID NO:14; SEQ ID NO:16 and SEQ ID NO:18. Amino acid sequence variants of the polypeptides of the present invention can be substitutional, insertional or deletion variants. Deletion variants lack one or more residues of the native protein that are not essential for function or immunogenic activity, and are exemplified by the variants lacking a transmembrane sequence. Another common type of deletion variant is one lacking secretory signal sequences or signal sequences directing a protein to bind to a particular part of a cell. Insertional mutants typically involve the addition of material at a non-terminal point in the polypeptide. This may include the insertion of an immunoreactive epitope or simply a single residue.

Substitutional variants typically contain the exchange of one amino acid for another at one or more sites within the protein, and may be designed to modulate one or more properties of the polypeptide, such as stability against proteolytic cleavage, without the loss of other functions or properties. Substitutions of this kind preferably are conservative, that is, one amino acid is replaced with one of similar shape and charge. Conservative substitutions are well known in the art and include, for example, the changes of: alanine to serine; arginine to lysine; asparagine to glutamine or histidine; aspartate to glutamate; cysteine to serine; glutamine to asparagine; glutamate to aspartate; glycine to proline; histidine to asparagine or glutamine; isoleucine to leucine or valine; leucine to valine or isoleucine; lysine to arginine; methionine to leucine or isoleucine; phenylalanine to tyrosine, leucine or methionine; serine to threonine; threonine to serine; tryptophan to tyrosine; tyrosine to tryptophan or phenylalanine; and valine to isoleucine or leucine.

The term biologically functional equivalent is well understood in the art and is further defined in detail herein. Accordingly, sequences that have between about 70% and about 80%; or more preferably, between about 81% and about 90%; or even more preferably, between about 91% and about 99%; of amino acids that are identical or functionally equivalent to the amino acids of a COMP polypeptide provided the biological activity of the protein is maintained.

The term functionally equivalent codon is used herein to refer to codons that encode the same amino acid, such as the six codons for arginine or serine, and also refers to codons that encode biologically equivalent amino acids (Table 1).

It also will be understood that amino acid and nucleic acid sequences may include additional residues, such as additional N- or C-terminal amino acids or 5′ or 3′ sequences, and yet still be essentially as set forth in one of the sequences disclosed herein, so long as the sequence meets the criteria set forth above, including the maintenance of biological protein activity where protein expression is concerned. The addition of terminal sequences particularly applies to nucleic acid sequences that may, for example, include various non-coding sequences flanking either of the 5′ or 3′ portions of the coding region or may include various internal sequences, i.e., introns, which are known to occur within genes.

The following is a discussion based upon changing of the amino acids of a protein to create an equivalent, or even an improved, second-generation molecule. For example, certain amino acids may be substituted for other amino acids in a protein structure without appreciable loss of interactive binding capacity with structures such as, for example, antigen-binding regions of antibodies or binding sites on substrate molecules. Since it is the interactive capacity and nature of a protein that defines that protein's biological functional activity, certain amino acid substitutions can be made in a protein sequence, and in its underlying DNA coding sequence, and nevertheless produce a protein with like properties. It is thus contemplated by the inventors that various changes may be made in the DNA sequences of genes without appreciable loss of their biological utility or activity, as discussed below. Table 1 shows the codons that encode particular amino acids.

In making such changes, the hydropathic index of amino acids may be considered. The importance of the hydropathic amino acid index in conferring interactive biologic function on a protein is generally understood in the art (Kyte and Doolittle, 1982). It is accepted that the relative hydropathic character of the amino acid contributes to the secondary structure of the resultant protein, which in turn defines the interaction of the protein with other molecules, for example, enzymes, substrates, receptors, DNA, antibodies, antigens, and the like.

It also is understood in the art that the substitution of like amino acids can be made effectively on the basis of hydrophilicity. U.S. Pat. No. 4,554,101, incorporated herein by reference, states that the greatest local average hydrophilicity of a protein, as governed by the hydrophilicity of its adjacent amino acids, correlates with a biological property of the protein. As detailed in U.S. Pat. No. 4,554,101, the following hydrophilicity values have been assigned to amino acid residues: arginine (+3.0); lysine (+3.0); aspartate (+3.0±1); glutamate (+3.0±1); serine (+0.3); asparagine (+0.2glutamine (+0.2); glycine (0); threonine (−0.4); proline (−0.5±1); alanine (−0.5); histidine (−0.5); cysteine (−1.0); methionine (−1.3); valine (−1.5); leucine (−1.8); isoleucine (−1.8); tyrosine (−2.3); phenylalanine (−2.5); tryptophan (−3.4).

It is understood that an amino acid can be substituted for another having a similar hydrophilicity value and still produce a biologically equivalent and immunologically equivalent protein. In such changes, the substitution of amino acids whose hydrophilicity values are within ±2 is preferred, those that are within ±1 are particularly preferred, and those within ±0.5 are even more particularly preferred.

As outlined herein, amino acid substitutions generally are based on the relative similarity of the amino acid side-chain substituents, for example, their hydrophobicity, hydrophilicity, charge, size, and the like. Exemplary substitutions that take into consideration the various foregoing characteristics are well known to those of skill in the art and include: arginine and lysine; glutamate and aspartate; serine and threonine; glutamine and asparagine; and valine, leucine and isoleucine.

Another embodiment for the preparation of polypeptides according to the invention is the use of peptide mimetics. Mimetics are peptide-containing molecules that mimic elements of protein secondary structure (Johnson 1993). The underlying rationale behind the use of peptide mimetics is that the peptide backbone of proteins exists chiefly to orient amino acid side chains in such a way as to facilitate molecular interactions, such as those of antibody and antigen. A peptide mimetic is expected to permit molecular interactions similar to the natural molecule. These principles may be used, in conjunction with the principles outline above, to engineer second generation molecules having many of the natural properties of adjuvants with altered and improved characteristics.

Other aspects of the present invention concern fusion proteins or peptides, which comprise the COMP domain linked or fused to an antigen domain. Such a fusion protein of the present invention may comprise all or a substantial portion of the COMP domain, linked at the amino terminus, to all or a portion of a antigen domain or an additional peptide, polypeptide, or protein such as a secretory region.

Other examples of fusion proteins involves the use of linkers which may comprise bifunctional cross-linking reagents. Such linkers are known to those of skill in the art. In addition, fusion proteins may comprise leader sequences from other species to permit the recombinant expression of a protein in a heterologous host. Another example of fusion proteins includes the addition of an immunologically active domain, such as an antibody epitope, to facilitate purification of the fusion protein. Inclusion of a cleavage site at or near the fusion junction facilitates removal of the extraneous polypeptide after purification.

Other useful fusions include linking of functional domains, such as active sites from enzymes, glycosylation domains, cellular targeting signals or transmembrane regions. Methods of generating fusion proteins are well known to those of skill in the art. For example, fusion proteins may be made by de novo synthesis of the complete fusion protein or by attachment of a nucleic acid sequence encoding the COMP domain to a nucleic acid sequence encoding the second peptide or protein such as a antigen domain, followed by expression of the intact fusion protein.

### IV. ANTIGENS AND ANTIGEN POLYPEPTIDES AND NUCLEIC ACIDS ENCODING THEM

The antigen domains of the present invention may be any protein or polypeptide sequence against which any form of immune response is desired. In general, antigens, are polypeptide sequences against which a humoral immune response can be raised.

The present invention encompasses methods of identifying antigenic proteins and polypeptide regions on a protein and methods of assaying and determining antigenicity and activity. The term “antigenic region” refers to a portion of a protein that is specifically recognized by an antibody or T-cell receptor. Antigenicity is relative to a particular organism. In many of the embodiments of the present invention, the organism is a human, but antigenicity may be discussed with respect to other organisms as well, such as other mammals—monkeys, gorillas, cows, rabbits, mice, sheep, cats, dogs, pigs, goats, etc.—as well as avian organisms and any other organism that can elicit an immune response.

There are many known antigenic polypeptides, and also many known methods of determining antigenic polypeptides. For example, antigens may be determined using the methodologies disclosed in U.S. Pat. No. 5,989,553, entitled “Expression Library Immunization” and/or in U.S. Pat. No. 6,410,241, entitled “Methods of screening open reading frames to determine whether they encode polypeptides with an ability to generate an immune response,” the entire contents and disclosures of which relating to any and all relevant techniques are hereby incorporated by reference herein for all purposes.

In some embodiments, polyclonal sera or monoclonal antibodies are employed with immunodetection methods to identify antigenic regions in a particular protein. Polyclonal sera may be collected from a variety of sources including workers suspected to have been occupationally exposed to a particular protein; patients suspected of or diagnosed as having a condition or disease that is accompanied or caused by the presence of antibodies to a particular protein or organism; patients who no longer have been treated for a condition or disease that is accompanied by the presence of antibodies to a particular protein or organism; and random subjects.

In some methods of the present invention, protein databases are employed after putative antigenic regions in a particular protein are identified. A region is then compared with a database containing protein sequences from the organism in which a lower immune response against the region is desired. A number of such databases exist both commercially and publicly, including GenBank, GenPept, SwissProt, PIR, PRF, PDB, all of which are available from the National Center for Biotechnology Information website.

Putative antigens may be tested for antigenicity using the techniques disclosed in this specification. Assays to determine antigenicity or activity of a protein include, but are not limited to immunodetection methods, and they are well known to those of skill in the art. Appropriate assays for a particular protein will vary depending on the protein. Enzymatic assays may be appropriate to evaluate the activity of an enzyme, for example. Further, where modified antigens are contemplated, one of skill in the art would be able to evaluate the activity of a modified protein relative to the native protein.

Once an antigenic protein of polypeptide region is identified, nucleic acids encoding it, whether native, modified, or synthesized may be employed in the context of the invention. These nucleic acid sequences may be obtained and employed in any manner known to those of skill in the art and/or disclosed herein.

### V. DELIVERY OF NUCLEIC ACIDS ENCODING COMP AND ANTIGENIC POLYPEPTIDES

Vectors have long been used to deliver nucleic acids to cells, these include viral vectors and non-viral vectors. As by methods described herein and as known to the skilled artisan, expression vectors in the present invention can be constructed to deliver nucleic acids encoding a COMP polypeptide and/or an antigen polypeptide to a cell, tissue, or an organism. These same methods are also useful to deliver nucleic acids encoding additional polypeptides to a cell, tissue, or organism For example, in the genetic immunization aspects of the invention, when a nucleic acid encoding an COMP polypeptide of the invention is being used as an adjuvant in conjunction with a nucleic acid encoding a polypeptide against which an immune response is desired, both nucleic acids may be administered in one or more vectors. In this case, the adjuvant nucleic acid and antigen encoding nucleic acid may be comprised on the same vector, or they may be comprised in separate vectors.

A vector in the context of the present invention refers to a carrier nucleic acid molecule into which a nucleic acid sequence encoding a polypeptide adjuvant can be inserted for introduction into a cell and thereby replicated. A nucleic acid sequence can be exogenous, which means that it is foreign to the cell into which the vector is being introduced; or that the sequence is homologous to a sequence in the cell but positioned within the host cell nucleic acid in which the sequence is ordinarily not found. Vectors include plasmids; cosmids; viruses such as bacteriophage, animal viruses, and plant viruses; and artificial chromosomes (e.g., YACs); and synthetic vectors such as linear/circular expression elements (LEE/CEE). One of skill in the art would be well equipped to construct a vector through standard recombinant techniques as described in Sambrook et al., 2001, Maniatis et al., 1990 and Ausubel et al., 1994, incorporated herein by reference.

An expression vector refers to any type of genetic construct comprising a nucleic acid coding for a RNA capable of being transcribed. In some cases, RNA molecules are then translated into a protein, polypeptide, or peptide. In other cases, these sequences are not translated, as in the case of antisense molecules or ribozymes production. Expression vectors can contain a variety of control sequences, which refer to nucleic acid sequences necessary for the transcription and possibly translation of an operably linked coding sequence in a particular host cell. In addition to control sequences that govern transcription and translation, vectors and expression vectors may contain nucleic acid sequences that serve other functions as well, and are described herein

A. Viral Vectors

There are a number of ways in which expression vectors may be introduced into cells. In certain embodiments of the invention, the expression vector comprises a virus or engineered vector derived from a viral genome. The ability of certain viruses to enter cells via receptor-mediated endocytosis, to integrate into host cell genome and express viral genes stably and efficiently have made them attractive candidates for the transfer of foreign genes into mammalian cells (Ridgeway, 1988; Nicolas and Rubinstein, 1988; Baichwal and Sugden, 1986; Temin, 1986). The first viruses used as gene vectors were DNA viruses including the papovaviruses (simian virus 40, bovine papilloma virus, and polyoma) (Ridgeway, 1988; Baichwal and Sugden, 1986) and adenoviruses (Ridgeway, 1988; Baichwal and Sugden, 1986). These have a relatively low capacity for foreign DNA sequences and have a restricted host spectrum. Furthermore, their oncogenic potential and cytopathic effects in permissive cells raise safety concerns. They can accommodate only up to 8 kb of foreign genetic material but can be readily introduced in a variety of cell lines and laboratory animals (Nicolas and Rubinstein, 1988; Temin, 1986).

The retroviruses are a group of single-stranded RNA viruses characterized by an ability to convert their RNA to double-stranded DNA in infected cells; they can also be used as vectors. Other viral vectors may be employed as expression constructs in the present invention. Vectors derived from viruses such as vaccinia virus (Ridgeway, 1988; Baichwal and Sugden, 1986; Coupar et al., 1988) adeno-associated virus (AAV) (Ridgeway, 1988; Baichwal and Sugden, 1986; Hermonat and Muzycska, 1984) and herpesviruses may be employed. They offer several attractive features for various mammalian cells (Friedmann, 1989; Ridgeway, 1988; Baichwal and Sugden, 1986; Coupar et al., 1988; Horwich et al., 1990).

Other viral vectors may be employed as constructs in the present invention. Vectors derived from viruses such as vaccinia virus (Ridgeway, 1988; Baichwal and Sugden, 1986; Coupar et al., 1988), sindbis virus, cytomegalovirus and herpes simplex virus may be employed. They offer several attractive features for various mammalian cells (Friedmann, 1989; Ridgeway, 1988; Baichwal and Sugden, 1986; Coupar et al., 1988; Horwich et al., 1990).

B. Linear and Circular Expression Elements

Linear or circular expression elements (LEEs/CEEs) technology allows for a rapid and effective means by which to determine the activity of a particular gene product or its physiological responses, by circumventing the use of plasmids and bacterial cloning procedures. In certain embodiments, the promoter and terminator sequences of the LEE/CEE may be regarded as a type of vector.

LEEs and/or CEEs may be made according to the disclosures of U.S. Pat. No. 6,410,241 and all related applications to it (U.S. patent appln. Ser. Nos. 10/077,508; 10/077,392; 10/077,247; 10/077,232; 10/077,621) are incorporated into this specification by reference.

Production of a LEE or circular expression element (CEE) generally comprise obtaining a nucleic acid segment comprising an open reading frame (ORF), and linking the ORF to a promoter, and a terminator, and/or other molecules such as a nucleic acid, to create LEE or CEE. The nucleic acid segment, terminator and/or additional nucleic acid(s) may be obtained by any method described herein or as would be known to one of ordinary skill in the art, including by nucleic acid amplification or chemical synthesis of nucleic acids such as described in EP 266,032, incorporated herein by reference, or as described by Froehler et al., 1986, and U.S. Pat. No. 5,705,629, each incorporated herein by reference.

### VI. DELIVERY OF NUCLEIC ACIDS ENCODING COMP POLYPEPTIDES AND/OR ANTIGEN POLYPEPTIDES

Suitable methods for delivery of nucleic acid encoding a COMP and/or antigen polypeptide for transformation of a cell, tissue, or organism for use with the current invention are believed to include virtually any method by which nucleic acids can be introduced into a cell, or an organism, as described herein or as would be known to one of ordinary skill in the art. Such methods include, but are not limited to: direct delivery of DNA by injection (U.S. Pat. Nos. 5,994,624, 5,981,274, 5,945,100, 5,780,448, 5,736,524, 5,702,932, 5,656,610, 5,589,466 and 5,580,859, each incorporated herein by reference), including microinjection (Harlan and Weintraub, 1985; U.S. Pat. No. 5,789,215, incorporated herein by reference); by electroporation (U.S. Pat. No. 5,384,253, incorporated herein by reference; Tur-Kaspa et al., 1986; Potter et al., 1984); by calcium phosphate precipitation (Graham and Van Der Eb, 1973; Chen and Okayama, 1987; Rippe et al., 1990); by using DEAE-dextran followed by polyethylene glycol (Gopal, 1985); by direct sonic loading (Fechheimer et al., 1987); by liposome mediated transfection (Nicolau and Sene, 1982; Fraley et al., 1979; Nicolau et al., 1987; Wong et al., 1980; Kaneda et al., 1989; Kato et al., 1991) and receptor-mediated transfection (Wu and Wu, 1987; Wu and Wu, 1988); by microprojectile bombardment (PCT Application Nos. WO 94/09699 and 95/06128; U.S. Pat. Nos. 5,610,042; 5,322,783 5,563,055, 5,550,318, 5,538,877 and 5,538,880, and each incorporated herein by reference); by agitation with silicon carbide fibers (Kaeppler et al., 1990; U.S. Pat. Nos. 5,302,523 and 5,464,765, each incorporated herein by reference); or by PEG-mediated transformation of protoplasts (Omirulleh et al., 1993; U.S. Pat. Nos. 4,684,611 and 4,952,500, each incorporated herein by reference); by desiccation/inhibition-mediated DNA uptake (Potrykus et al., 1985), and any combination of such methods. Through the application of techniques such as these, organelle(s), cell(s), tissue(s) or organism(s) may be stably or transiently transformed. In certain embodiments, acceleration methods are preferred and include, for example, microprojectile bombardment.

VII. Pharmacological Preparations of Nucleic Acids Encoding COMP and/or an Antigen

A. Routes of Delivery/Administration

The preparation of vaccines which contain peptides or nucleic acids encoding peptides as active ingredients is generally well understood in the art, as exemplified by U.S. Pat. Nos. 4,608,251; 4,601,903; 4,599,231; 4,599,230; 4,596,792; and 4.578,770, all incorporated herein by reference. Typically, such vaccines are prepared as injectables, either as liquid solutions or suspensions or solid forms suitable for solution in, or suspension in, liquid prior to injection. The preparation may also be emulsified. The active immunogenic ingredient is often mixed with excipients which are pharmaceutically acceptable and compatible with the active ingredient. Suitable excipients are, for example, water, saline, dextrose, glycerol, ethanol, or the like and combinations thereof. In addition, if desired, the vaccine may contain minor amounts of auxiliary substances such as wetting or emulsifying agents, pH buffering agents, or adjuvants which enhance the effectiveness of the vaccines.

Vaccines may be conventionally administered parenterally, by injection, for example, either subcutaneously or intramuscularly.

The manner of application may be varied widely. Any of the conventional methods for administration of a vaccine are applicable. These are believed to include gene gun inoculation of the DNA encoding the antigen peptide(s), phage transfection of the DNA, oral application on a solid physiologically acceptable base or in a physiologically acceptable dispersion, parenterally, by injection or the like. The dosage of the vaccine will depend on the route of administration and will vary according to the size of the host.

Various methods of achieving adjuvant effect for the vaccine includes use of agents such as aluminum hydroxide or phosphate (alum), commonly used as about 0.05 to about 0.1% solution in phosphate buffered saline, admixture with synthetic polymers of sugars (Carbopol®) used as an about 0.25% solution, aggregation of the protein in the vaccine by heat treatment with temperatures ranging between about 70° to about 101° C. for a 30-second to 2-minute period, respectively. Aggregation by reactivating with pepsin treated (Fab) antibodies to albumin, mixture with bacterial cells such as C. parvum or endotoxins or lipopolysaccharide components of Gram-negative bacteria, emulsion in physiologically acceptable oil vehicles such as mannide mono-oleate (Aracel A) or emulsion with a 20% solution of a perfluorocarbon (Fluosol-DA®) used as a block substitute may also be employed.

B. Administration of nucleic acids

One method for the delivery of a nucleic acid encoding a COMP domain and/or antigenic domain as in the present invention is via gene gun injection. As known to the skilled artisan, the two main methods of administration of DNA vaccines are via particle bombardment, achieved using a gene gun, or via intramuscular administration. For the gene gun method as employed by the present invention, the DNA is coated onto gold particles which are then fired into the target tissue which is usually the epidermis. Gene gun methods have been shown to be the most efficient as the same level of antibody and cellular immunity may be gained using 100-5000 fold less DNA than is necessary for injection methods (Pertmer et al., 1995; Fynan et al., 1993). Although the gene gun method is more efficient it has not been shown to have longer lived responses or provide better protection from pathogenic challenge than intramuscular vaccination (Cohen et al., 1998). The interesting difference between the two methods is that they elicit different Th responses. The intramuscular inoculation is associated with a Th-1 response producing elevated interferon gamma, little IL-4 and more IgG2a than IgGI antibodies (Pertmer et al., 1996). The gene gun method, on the other hand, produces a Th-2 response, on successive immunizations, with the opposite cytokine and antibody profile to the intramuscular inoculation. However, the pharmaceutical compositions disclosed herein may alternatively be administered parenterally, intravenously, intradermally, intramuscularly, transdermally or even intraperitoneally as described in U.S. Pat. No. 5,543,158; U.S. Pat. No. 5,641,515 and U.S. Pat. No. 5,399,363 (each specifically incorporated herein by reference in its entirety).

Injection of a nucleic acid encoding a COMP domain and/or antigen may be delivered by syringe or any other method used for injection of a solution, as long as the expression construct can pass through the particular gauge of needle required for injection. A novel needleless injection system has recently been described (U.S. Pat. No. 5,846,233) having a nozzle defining an ampule chamber for holding the solution and an energy device for pushing the solution out of the nozzle to the site of delivery. A syringe system has also been described for use in gene therapy that permits multiple injections of predetermined quantities of a solution precisely at any depth (U.S. Pat. No. 5,846,225).

Solutions of the active compounds as free base or pharmacologically acceptable salts may be prepared in water suitably mixed with a surfactant, such as hydroxypropylcellulose. Dispersions may also be prepared in glycerol, liquid polyethylene glycols, and mixtures thereof and in oils. Under ordinary conditions of storage and use, these preparations contain a preservative to prevent the growth of microorganisms. The pharmaceutical forms suitable for injectable use include sterile aqueous solutions or dispersions and sterile powders for the extemporaneous preparation of sterile injectable solutions or dispersions (U.S. Pat. No. 5,466,468, specifically incorporated herein by reference in its entirety). In all cases the form must be sterile and must be fluid to the extent that easy syringability exists. It must be stable under the conditions of manufacture and storage and must be preserved against the contaminating action of microorganisms, such as bacteria and fungi. The carrier can be a solvent or dispersion medium containing, for example, water, ethanol, polyol (e.g., glycerol, propylene glycol, and liquid polyethylene glycol, and the like), suitable mixtures thereof, and/or vegetable oils. Proper fluidity may be maintained, for example, by the use of a coating, such as lecithin, by the maintenance of the required particle size in the case of dispersion and by the use of surfactants. The prevention of the action of microorganisms can be brought about by various antibacterial and antifungal agents, for example, parabens, chlorobutanol, phenol, sorbic acid, thimerosal, and the like. In many cases, it will be preferable to include isotonic agents, for example, sugars or sodium chloride. Prolonged absorption of the injectable compositions can be brought about by the use in the compositions of agents delaying absorption, for example, aluminum monostearate and gelatin.

For parenteral administration in an aqueous solution, for example, the solution should be suitably buffered if necessary and the liquid diluent first rendered isotonic with sufficient saline or glucose. These particular aqueous solutions are especially suitable for intravenous, intramuscular, subcutaneous, intratumoral and intraperitoneal administration. In this connection, sterile aqueous media that can be employed will be known to those of skill in the art in light of the present disclosure. For example, one dosage may be dissolved in 1 ml of isotonic NaCl solution and either added to 1000 ml of hypodermoclysis fluid or injected at the proposed site of infusion, (for example, “Remington's Pharmaceutical Sciences” 15th Edition, pages 1035-1038 and 1570-1580). Some variation in dosage will necessarily occur depending on the condition of the subject being treated. The person responsible for administration will, in any event, determine the appropriate dose for the individual subject. Moreover, for human administration, preparations should meet sterility, pyrogenicity, general safety and purity standards as required by FDA Office of Biologics standards.

Sterile injectable solutions are prepared by incorporating the active compounds in the required amount in the appropriate solvent with various of the other ingredients enumerated above, as required, followed by filtered sterilization. Generally, dispersions are prepared by incorporating the various sterilized active ingredients into a sterile vehicle which contains the basic dispersion medium and the required other ingredients from those enumerated above. In the case of sterile powders for the preparation of sterile injectable solutions, the preferred methods of preparation are vacuum-drying and freeze-drying techniques which yield a powder of the active ingredient plus any additional desired ingredient from a previously sterile-filtered solution thereof.

The compositions disclosed herein may be formulated in a neutral or salt form. Pharmaceutically-acceptable salts, include the acid addition salts (formed with the free amino groups of the protein) and which are formed with inorganic acids such as, for example, hydrochloric or phosphoric acids, or such organic acids as acetic, oxalic, tartaric, mandelic, and the like. Salts formed with the free carboxyl groups can also be derived from inorganic bases such as, for example, sodium, potassium, ammonium, calcium, or ferric hydroxides, and such organic bases as isopropylamine, trimethylamine, histidine, procaine and the like. Upon formulation, solutions will be administered in a manner compatible with the dosage formulation and in such amount as is therapeutically effective. The formulations are easily administered in a variety of dosage forms such as injectable solutions, drug release capsules and the like.

As used herein, carrier includes any and all solvents, dispersion media, vehicles, coatings, diluents, antibacterial and antifungal agents, isotonic and absorption delaying agents, buffers, carrier solutions, suspensions, colloids, and the like. The use of such media and agents for pharmaceutical active substances is well known in the art. Except insofar as any conventional media or agent is incompatible with the active ingredient, its use in the therapeutic compositions is contemplated. Supplementary active ingredients can also be incorporated into the compositions.

The phrase “pharmaceutically-acceptable” or “pharmacologically-acceptable” refers to molecular entities and compositions that do not produce an allergic or similar untoward reaction when administered to a human. The preparation of an aqueous composition that contains a protein as an active ingredient is well understood in the art. Typically, such compositions are prepared as injectables, either as liquid solutions or suspensions; solid forms suitable for solution in, or suspension in, liquid prior to injection can also be prepared.

A vaccination schedule and dosages may be varied on a subject by subject basis, taking into account, for example, factors such as the weight and age of the subject, the type of disease being treated, the severity of the disease condition, previous or concurrent therapeutic interventions, the manner of administration and the like, which can be readily determined by one of ordinary skill in the art.

A vaccine is administered in a manner compatible with the dosage formulation, and in such amount as will be therapeutically effective and immunogenic. For example, the intramuscular route may be preferred in the case of toxins with short half lives in vivo. The quantity to be administered depends on the subject to be treated, including, e.g., the capacity of the individual's immune system to synthesize antibodies, and the degree of protection desired. The dosage of the vaccine will depend on the route of administration and will vary according to the size of the host. Precise amounts of an active ingredient required to be administered depend on the judgment of the practitioner. In certain embodiments, pharmaceutical compositions may comprise, for example, at least about 0.1% of an active compound. In other embodiments, the an active compound may comprise between about 2% to about 75% of the weight of the unit, or between about 25% to about 60%, for example, and any range derivable therein However, a suitable dosage range may be, for example, of the order of several hundred micrograms active ingredient per vaccination. In other non-limiting examples, a dose may also comprise from about 1 microgram/kg/body weight, about 5 microgram/kg/body weight, about 10 microgram/kg/body weight, about 50 microgram/kg/body weight, about 100 microgram/kg/body weight, about 200 microgram/kg/body weight, about 350 microgram/kg/body weight, about 500 microgram/kg/body weight, about 1 milligram/kg/body weight, about 5 milligram/kg/body weight, about 10 milligram/kg/body weight, about 50 milligram/kg/body weight, about 100 milligram/kg/body weight, about 200 milligram/kg/body weight, about 350 milligram/kg/body weight, about 500 milligram/kg/body weight, to about 1000 mg/kg/body weight or more per vaccination, and any range derivable therein. In non-limiting examples of a derivable range from the numbers listed herein, a range of about 5 mg/kg/body weight to about 100 mg/kg/body weight, about 5 microgram/kg/body weight to about 500 milligram/kg/body weight, etc., can be administered, based on the numbers described above. A suitable regime for initial administration and booster administrations (e.g., inoculations) are also variable, but are typified by an initial administration followed by subsequent inoculation(s) or other administration(s).

In many instances, it will be desirable to have multiple administrations of the vaccine, usually not exceeding six vaccinations, more usually not exceeding four vaccinations and preferably one or more, usually at least about three vaccinations. The vaccinations will normally be at from two to twelve week intervals, more usually from three to five week intervals. Periodic boosters at intervals of 1-5 years, usually three years, will be desirable to maintain protective levels of the antibodies.

The course of the immunization may be followed by assays for antibodies for the supernatant antigens. The assays may be performed by labeling with conventional labels, such as radionuclides, enzymes, fluorescents, and the like. These techniques are well known and may be found in a wide variety of patents, such as U.S. Pat. Nos. 3,791,932; 4,174,384 and 3,949,064, as illustrative of these types of assays. Other immune assays can be performed and assays of protection from challenge can be performed, following immunization.

### VIII. EXAMPLES

The following examples are included to demonstrate preferred embodiments of the invention. It should be appreciated by those of skill in the art that the techniques disclosed in the examples which follow represent techniques discovered by the inventor to function well in the practice of the invention, and thus can be considered to constitute preferred modes for its practice. However, those of skill in the art should, in light of the present disclosure, appreciate that many changes can be made in the specific embodiments which are disclosed and still obtain a like or similar result without departing from the spirit and scope of the invention.

### Example 1

### Materials and Methods

The following materials and methods were used for Examples 2-4 below.

Construction of plasmids. The genetic immunization plasmids were derived from pCAGGS (Niwa et al., 1991). The inventors replaced the human cytomegalovirus (CMV) promoter with a synthetic promoter SP72. The SP72 element was designed de novo from consensus binding sites for transcription factors and rivals CMV in terms of producing antibody responses (B. Qu, personal communication). A 618 bp fragment containing the SP72 promoter was sub-cloned at the SalI and EcoRI sites, thereby replacing the CMV promoter and intron, creating pSP72. Gene synthesis was used to construct a 346 bp DNA fragment containing in the following order; an EcoRI site, a consensus translation initiation site, the leader sequence from AAT, the antigenic tag, COMP and restriction sites for BclI, XmaI and XbaI. The fragment was digested with EcoRI and XbaI and sub-cloned into the same sites in pSP72 to create pBQAP10. The plasmid pCMVi10 was identical except retained the original CMV promoter and intron. The plasmids pBQAP-OVA, pBQAP-TT were based on pBQAP10 and were created by sub-cloning a BglII and XmaI digested DNA fragment encoding the T cell epitopes, and created by gene synthesis, into the BclI and XmaI sites. A new BclI site was designed after the T cell epitope coding regions. The plasmid pGST-FRP was derived from pGST-CS (Chang et al., 2001) by sub-cloning a pair of annealed oligonucleotides at the NcoI and EcoRI sites. This replaced the existing multiple cloning sites for BglII, BamHI and XmaI. The expression plasmids encoding GM-CSF and Flt3L were constructed by sub-cloning mouse cDNAs into pCMVi-SS (Sykes and Johnston, 1999) at the BglII and KpnI sites.

Gene synthesis. Genes were designed with a set of codons selected for efficient expression in both mice and E. coli using the codon-optimizing software, DNA Builder (http://cbi.swmed.edu/computation/cbu/dnabuilder.html), and for design flexibility to avoid hairpins and other inappropriate matches amongst the sequence that can hinder gene synthesis. The codons used were as follows: Ala; GCA (33%), GCT (33%), GCC (34%), Cys; TGT (50%), TGC (50%), Asp; GAT (50%), GAC (50%), Glu; GAG (50%), GAA (50%), Phe; TTT (25%), TTC (75%), Gly; GGT (50%), GGC (50%), His; CAT (25%), CAC (75%), Ile; ATT (25%), ATC (75%), Lys; AAG (50%), AAA (50%), Leu; CTG (100%), Met; ATG (100%), Asn; AAC (100%), Pro; CCG (50%), CCA (50%), Gln; CAG (75%), CAA (25%), Arg; CGT (25%), CGC (75%), Ser; TCT (50%), AGC (50%), Thr; ACT (50%), ACC (50%), Val; GTG (75%), GTT (25%), Trp; TGG (100%), Tyr; TAT (50%), TAC (50%). A set of overlapping oligonucleotides were designed using the custom software DNABuilder. The software can be downloaded at http://cbi.swmed.edu/computation/cbu. The oligonucleotides were assembled into a DNA fragment using PCR™ (Stemmer et al., 1995). Genes were sub-cloned into the appropriate plasmids and sequenced to identify a correct clone. Mutations occurred at a frequency of 0.3%.

UDG cloning. PCR™ products were generated using primers containing 5′ flanks as previously described (Smith et al., 1993). The forward primers contained the flanking sequence; ATAUCGAUAUCGAUGAU (SEQ ID NO:21), and the reverse primers contained the flanking sequence; AGUGAUCGAUGCATUACU (SEQ ID NO:22). Vector preparations were created by digesting the plasmids with BclI and XmaI (PBQAP10, pBQAP-OVA, pBQAP-TT), or BglII and XmaI (pGST-FRP), and ligating the following oligonucleotides to the 4 bp overhangs; GATCATATCGATATCGATGAT (SEQ ID NO:23) and CCGGAGTGATCGATGCATTACT (SEQ ID NO:24). PCR™ products are sub-cloned by mixing 50 ng of the vector preparation with 10 ng of the PCR™ product in the presence of 0.5 units of uracil DNA glycosylase (New England Biolabs), 10 mM Tris-HCl pH 7.9, 10 mM MgCl₂, 50 mM NaCl, and 1 mM DTT in a final volume of 10 μl. Reactions were incubated at 37° C. for 30 min and 1 μl was used to transform E. coli DH10B.

Genetic immunization and analyses. All procedures for handling mice were approved by the UT Southwestern Medical Center IACRAC. Plasmids were delivered using the Helios gene gun (Biorad). Bullets were prepared as per the manufacturers instructions with a mixture of plasmid encoding the antigen and plasmids encoding mouse GM-CSF and mouse Flt3L (2:1:1 ratio). Each bullet contained approximately 1 μg of DNA. Mice were anesthetized with avertin (0.4 ml/20 g mouse) and shot in each ear using 400 psi to fire the gene gun. Blood was collected via tail bleeds, allowed to stand for 2 h at room temperature and the sera collected by centrifugation. Western blots and ELISAs were performed as described previously (Sykes and Johnston, 1999; Chambers and Johnston, 2003). Each ELISA was performed using a AAT monoclonal antibody as a standard (Calbiochem) to calculate antibody equivalents in μg/ml. Titers were defined as the reciprocal of the sera dilution that produced a signal 2-fold above background (age matched sera). GST fusion proteins were generated in E. coli strain DH10B by inducing 2 ml log phase cultures with IPTG. Whole cell extracts were prepared from bacteria two hours after induction. Cells were pelleted, resuspended in 200 μl of PBS, mixed with 200 μl of SDS lysis buffer and heated for 5 min at 95° C.

### Example 2

### Design of the pBQAP10 Genetic Immunization Vector

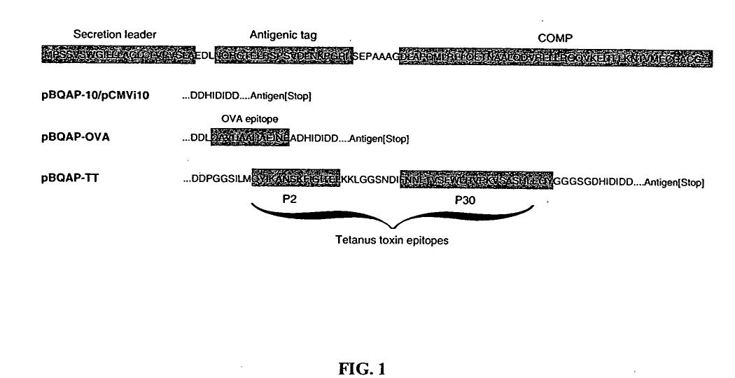

A specialized genetic immunization plasmid, pBQAP10, was created for the purpose of generating antibodies (FIG. 1 (SEQ ID NO:27; SEQ ID NO:28; SEQ ID NO:29; SEQ ID NO:30; SEQ ID NO:31)). The plasmid encodes a secretion leader sequence from the highly expressed human α1-antitrypsin (AAT) gene. Many studies have demonstrated that adding a secretion leader sequence can dramatically increase the antibody response (Svanholm et al., 1999; Li et al., 1999). Following the leader sequence is a unique 20 amino acid antigenic tag that the inventors included as an internal control. Secretion of the antigen may be blocked by ‘quality control’ if it is poorly folded and/or insoluble (Hammond and Helenius, 1995). To help overcome this potential problem the inventors included a highly soluble and stably-folded domain from the rat cartilage oligomerization matrix protein (COMP) (Terskikh et al., 1997). The 46 residue COMP domain can also form pentamers and may enhance antigen uptake by antigen presenting cells and/or allow T-help independent B cell activation (St. Clair et al., 1999; Valenzuela et al., 1982).

### Example 3

### Antibody Response of Mice Immunized with pBQAP10-AAT

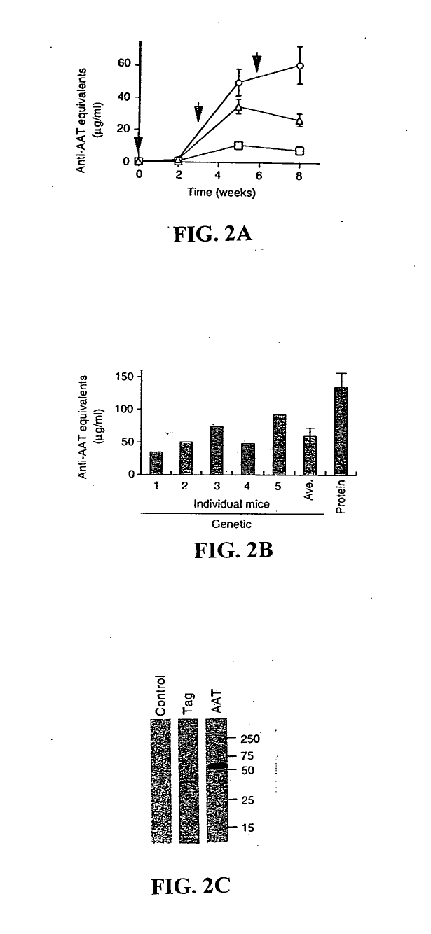

The human AAT gene was used as an antigen to test the efficacy of pBQAP10 in genetic immunization. Many different cytokines have previously been tested as genetic adjuvants, with mixed results (Scheerlinck, 2001). GM-CSF-expressing plasmids have been widely used in genetic immunization studies and almost always results in an increase in antibody titer (Scheerlinck, 2001). GM-CSF is a potent growth factor for dendritic cells, although its exact mechanism of action in genetic immunization is poorly understood. Mice were immunized with the AAT encoded plasmid using a gene gun, either with or without co-administration of plasmids encoding the cytokines GM-CSF and Flt3L. ELISA measurements of sera showed that the mice co-immunized with both the GM-CSF and Flt3L plasmids had approximately a 9-fold higher level of antibodies (3×10⁴ titer, FIG. 2A). For comparison, a group of mice immunized conventionally using AAT protein with Freund's complete adjuvant produced antibody titers of 7×10⁴. All genetically immunized mice responded with relatively little variation in levels (FIG. 2B). Isotyping of the AAT antibodies showed only the IgG1 isotype (data not shown). The specificity of the sera was tested by probing a western blot containing AAT mixed with an E. coli whole cell extract. Pooled sera from five mice recognized a single band of the correct size for AAT (FIG. 2C).

To evaluate the general utility of this antibody production system, the inventors tested it using a set of 100 antigen genes (Table 1). Of the 100 genes tested, 36% encoded fragments of the mature form of the protein. The average identity of the human antigens to mouse proteins was 76%, and the average antigen size was 179 residues. Most of the genes were of human origin and the inventors explored three general sources of antigen genes; genomic DNA (20), cDNA (52), and gene synthesis from oligonucleotides (28). In principle, amplifying genes from genomic DNA is the simplest approach since only a single template and two PCR™ primers are required per gene, or four primers for nested PCR™. Genes fragmented into small exons may present a problem. For example genes in the human genome are on average broken into 8.8 exons encoding an average length of 50 residues (International Human Genome Sequencing Consortium, 2001). Using cDNA would bypass this problem but is more difficult logistically. Both genomic DNA and cDNA have the disadvantage in that the genes may contain suboptimal codon usage. Codon optimization of genes has been shown to dramatically increase translation, and as a consequence, antibody responses (Andre et al., 1998; Stratford et al., 2001). Gene synthesis allows codons to be optimized for expression and gives unrestricted access to any gene sequence. Genes were recoded using a subset of codons allowing efficient expression in both mice and E. coli (see Methods).

TABLE 1
List of Antigens Tested in pBQAP10/pCMVi10

| | | | | Size | | |
|---|---|---|---|---|---|---|
| | Antigen Name | Accession | Homology | (bp) | Source | Response |
| 1 | AAT | X01683 | 64% | 1101 | cDNA | + |
| 2 | ApoAV | NM052968 | 72% | 300 | Synthetic | + |
| 3 | ApoA1 | X00566 | 65% | 732 | cDNA | + |
| 4 | ApoCIV | T71886 | 56% | 381 | cDNA | − |
| 5 | ApoD | H15842 | 73% | 429 | cDNA | + |
| 6 | Aquaporin 4 | N46843 | 93% | 399 | cDNA | − |
| 7 | ARF1 | M84326 | 100% | 549 | cDNA | + |
| 8 | Calpain I | H15456 | 89% | 399 | cDNA | + |
| 9 | CaMK4 | AW025962 | 80% | 399 | cDNA | + |
| 10 | CDC42 | M57298 | 100% | 570 | cDNA | − |
| 11 | CDK9 | X80230 | 98% | 300 | Synthetic | − |
| 12 | Cyp 7B1 | AF127090 | 66% | 288 | Genomic DNA | − |
| 13 | EGF | X04571 | 67% | 159 | Synthetic | + |
| 14 | Endothelin 1 | J05008 | 70% | 639 | cDNA | + |
| 15 | FABP1, liver | T53220 | 84% | 384 | cDNA | + |
| 16 | FACT, p140 | NM007192 | 98% | 300 | Synthetic | − |
| 17 | FGFβ | M27968 | 94% | 465 | Synthetic | − |
| 18 | FGL2 | Z36531 | 77% | 612 | Genomic DNA | + |
| 19 | FKBP 1A | M34539 | 97% | 321 | cDNA | + |
| 20 | Gα s long | X04409 | 94% | 1179 | cDNA | − |
| 21 | Gγ 1 | S62027 | 96% | 219 | cDNA | + |
| 22 | GMCSF | M11230 | 80% | 435 | cDNA | + |
| 23 | GRB2 | X62852 | 99% | 651 | cDNA | − |
| 24 | GROα | J03561 | 62% | 237 | Synthetic | + |
| 25 | HDAC5 | NM005474 | 94% | 918 | cDNA | + |
| 26 | Interferonα | J00210 | 64% | 498 | Synthetic | + |
| 27 | Interferonγ | X13274 | 41% | 438 | Synthetic | + |
| 28 | Interleukin 1α | X02531 | 61% | 477 | Synthetic | + |
| 29 | Interleukin 1β | M15330 | 68% | 510 | cDNA | + |
| 30 | Interleukin 10 | M57627 | 73% | 429 | cDNA | − |
| 31 | Interleukin 2 | X01586 | 63% | 399 | Synthetic | − |
| 32 | Interleukin 3 | M17115 | 31% | 399 | Synthetic | + |
| 33 | Interleukin 4 | M13982 | 41% | 387 | Synthetic | + |
| 34 | Interleukin 5 | X04688 | 70% | 336 | Synthetic | − |
| 35 | Interleukin 6 | M14584 | 41% | 459 | cDNA | − |
| 36 | Interleukin 7 | J04156 | 61% | 456 | Synthetic | + |
| 37 | Interleukin 8 | M28130 | 47% | 240 | cDNA | + |
| 38 | Interleukin 9 | M30134 | 56% | 378 | Synthetic | + |
| 39 | Leptin | U43653 | 83% | 438 | Synthetic | − |
| 40 | Lipase-HS | W96325 | 85% | 399 | cDNA | + |
| 41 | MCIP1 | U28833 | 96% | 594 | cDNA | + |
| 42 | MCP1 | X14768 | 67% | 231 | Synthetic | + |
| 43 | MDM2 | M92424 | 80% | 564 | cDNA | + |
| 44 | MIP1α | M23452 | 76% | 216 | Synthetic | + |
| 45 | MLCK1 | U48959 | 33% | 219 | Synthetic | − |
| 46 | MLCK2 | U48959 | 33% | 300 | Synthetic | + |
| 47 | Myoglobin | X00371 | 83% | 465 | cDNA | + |
| 48 | Myosin light | N93941 | 92% | 399 | cDNA | + |
| | chain 2a | | | | | |
| 49 | NFKB, p65 | L19067 | 100% | 309 | cDNA | + |
| 50 | NGFβ | NM002506 | 83% | 399 | Synthetic | + |
| 51 | OS-9 | AA013336 | 21% | 399 | cDNA | + |
| 52 | Phospholamban | M63603 | 98% | 159 | cDNA | + |
| 53 | Pirin | H69334 | 95% | 399 | cDNA | − |
| 54 | RALA | X15014 | 99% | 630 | cDNA | − |
| 55 | RANTES | M21121 | 80% | 204 | Synthetic | + |
| 56 | RGS1 | X73427 | 87% | 591 | cDNA | + |
| 57 | Rho GDIα | D13989 | 68% | 609 | cDNA | + |
| 58 | RPB1-CTD | X63564 | 99% | 210 | Synthetic | + |
| 59 | Rv 0105c (Mtb) | NC000962 | — | 282 | Genomic DNA | − |
| 60 | Rv 0358 (Mtb) | NC000962 | — | 645 | Genomic DNA | − |
| 61 | Rv 0928 (Mtb) | NC000962 | — | 1110 | Genomic DNA | + |
| 62 | Rv 1386 (Mtb) | NC000962 | — | 306 | Genomic DNA | + |
| 63 | Rv 1813c (Mtb) | NC000962 | — | 429 | Genomic DNA | + |
| 64 | Rv 2031c (Mtb) | NC000962 | — | 432 | Genomic DNA | + |
| 65 | Rv 2703 (Mtb) | NC000962 | — | 1584 | Genomic DNA | + |
| 66 | Rv 3286c (Mtb) | NC000962 | — | 783 | Genomic DNA | + |
| 67 | Rv 3314c (Mtb) | NC000962 | — | 1281 | Genomic DNA | + |
| 68 | Rv 3415c (Mtb) | NC000962 | — | 825 | Genomic DNA | − |
| 69 | Rv 3477 (Mtb) | NC000962 | — | 294 | Genomic DNA | + |
| 70 | Rv 3614c (Mtb) | NC000962 | — | 552 | Genomic DNA | + |
| 71 | Rv 3773c (Mtb) | NC000962 | — | 582 | Genomic DNA | + |
| 72 | Rv 3904c (Mtb) | NC000962 | — | 270 | Genomic DNA | − |
| 73 | RXRβ | M84820 | 94% | 234 | Genomic DNA | + |
| 74 | SC/MCGF | NM000899 | 82% | 399 | Synthetic | − |
| 75 | SCYA16 | T58775 | 39% | 363 | cDNA | + |
| 76 | SOD | X02317 | 83% | 465 | cDNA | + |
| 77 | TAF250 | D90359 | 36% | 300 | Synthetic | + |
| 78 | TBP | X54993 | 91% | 300 | Synthetic | − |
| 79 | TGFβ | X02812 | 89% | 336 | Synthetic | − |
| 80 | Tropomyosin 2 | AA477400 | 98% | 390 | cDNA | + |
| 81 | Troponin C | X07897 | 99% | 483 | cDNA | + |
| 82 | Troponin I | X90780 | 93% | 471 | cDNA | + |
| 83 | Troponin T2 | N70734 | 85% | 399 | cDNA | − |
| 84 | UCP1 | U28480 | 79% | 198 | Genomic DNA | − |
| 85 | UCP2 | U94592 | 96% | 180 | Genomic DNA | + |
| 86 | USF1 | X55666 | 98% | 195 | Genomic DNA | + |
| 87 | VEGF-D | AA995128 | 83% | 399 | cDNA | + |
| 88 | ZIF38 | AC025271 | — | 399 | cDNA | + |

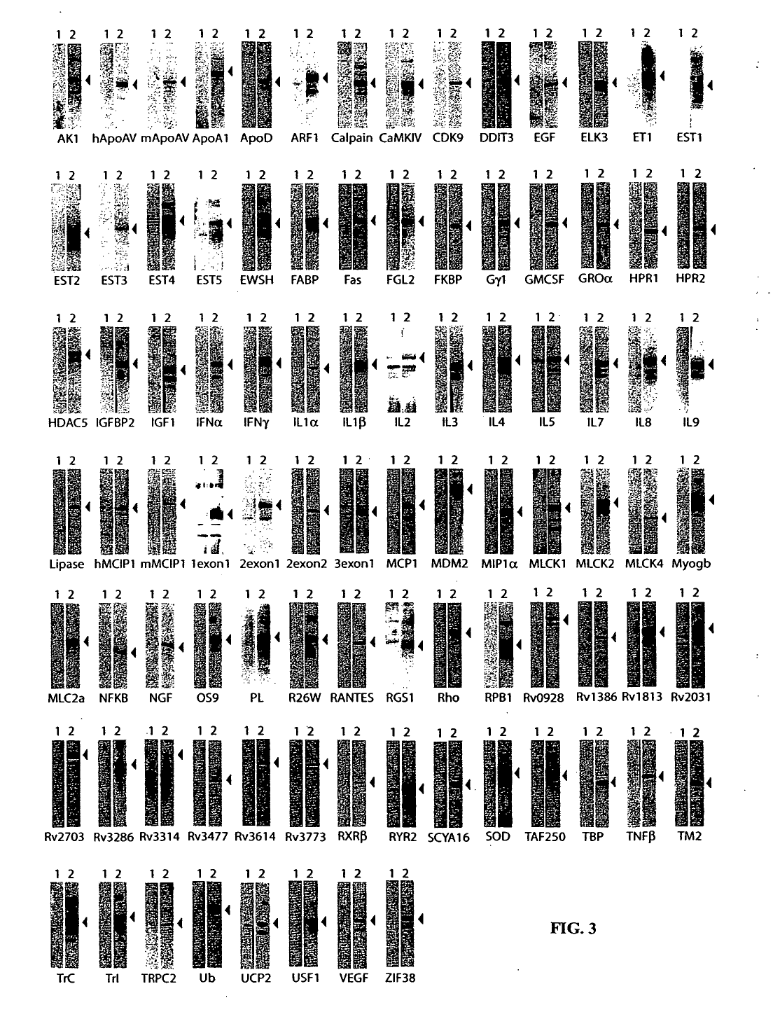

PCR™ products of the 100 antigen genes were generated using primers with a flanking sequence containing deoxyuracil (dU) residues allowing rapid cloning (Smith et al., 1993). The genes were cloned into pBQAP10 (80) or pCMVi10 (20) to allow genetic immunization of mice and pGST-FRP for overexpression in E. coli. Eighty-eight of the 100 proteins successfully overexpressed in E. coli. Groups of two CD 1 mice were immunized and were boosted every three weeks until a total of four shots had been administered. Sera from mice were tested every three weeks by western blotting and were scored successful if it could detect 50 ng of the antigen at sera dilutions of 1:5000. Antibodies were detected against 62 of the 88 test antigens (70%) and were produced after an average of two immunizations (Table 1 and FIG. 3). The pBQAP10 and pCMVi10 vectors had similar efficacies.

Antigens that have high identity to sequences from the immunized host typically do not produce an antibody response due to tolerance mechanisms (Zinkernagel, 2000). Analysis of the antigens tested in pBQAP10/pCMVi10 indicated this may indeed be a limiting factor, since antigens that failed to produce an antibody response had on average a higher identity to a mouse protein than successful antigens (69% versus 61%; Table 1). Humoral tolerance can be overcome by adding exogenous T cell epitopes fused to the antigen (King et al., 1998; Dalum et al., 1996). To evaluate this idea the inventors created two new vectors, pBQAP-TT and pBQAP-OVA (FIG. 1 (SEQ ID NO:27; SEQ ID NO:28; SEQ ID NO:29; SEQ ID NO:30; SEQ ID NO:32; SEQ ID NO:33; SEQ ID NO:34; SEQ ID NO:35; SEQ ID NO:36)), that contained either the P2 and P30 ‘universal’ T cell epitopes and flanking regions from tetanus toxin (50 residues), or the ovalbumin (325-336) T cell epitope (12 residues).

A set of 38 gene fragments were cloned into either pBQAP-TT or pBQAP-OVA (Table 2). Most of the genes encoded proteins that were expected to be poorly antigenic, either because they were small (≦20 amino acids), highly identical to mouse sequences (up to 100%), or had previously failed using protein-based immunizations. In addition, the inventors included five genes that previously failed to yield antibodies in genetic immunizations when cloned in pBQAP10. The target region of each gene was selected based on its antigenicity index score (Jameson and Wolf, 1988). On average, the antigens contained 73 amino acids and had a 90% identity to a mouse protein.

TABLE 2
List of Antigens Tested in pBQAP-OVA/pBQAP-TT.

| | | | | Size | | | |
|---|---|---|---|---|---|---|---|
| | Name | Accession | Homology | (bp) | Source | Epitope | Response |
| 1 | ADR | S56143 | 93% | 30 | synthetic | OVA | − |
| 2 | AK1 (mouse) | BG795557 | 100% | 300 | synthetic | OVA | + |
| 3 | ApoAV (mouse) | NM080434 | 100% | 300 | synthetic | TT | + |
| 4 | CDK9 | X80230 | 98% | 300 | synthetic | TT | + |
| 5 | DDIT3 (mouse) | AA914803 | 100% | 300 | synthetic | OVA | + |
| 6 | ELK3 (mouse) | NM013508 | 100% | 300 | synthetic | OVA | + |
| 7 | EST1 (mouse) | BG795231 | 100% | 300 | synthetic | OVA | + |
| 8 | EST2 (mouse) | BG795231 | 100% | 54 | synthetic | OVA | + |
| 9 | EST3 (mouse) | BG795399 | 100% | 42 | synthetic | OVA | + |
| 10 | EST4 (mouse) | AA511850 | 100% | 300 | synthetic | OVA | + |
| 11 | EST5 (mouse) | AA512810 | 100% | 300 | synthetic | OVA | + |
| 12 | EWSH (mouse) | BG795113 | 100% | 300 | synthetic | OVA | + |
| 13 | Fas (mouse) | M83649 | 100% | 300 | synthetic | TT | + |
| 14 | GBL (mouse) | NM019988 | 100% | 300 | synthetic | OVA | − |
| 15 | HPR (region 1) | X89214 | 75% | 60 | synthetic | TT | + |
| 16 | HPR(region 2) | X89214 | 75% | 60 | synthetic | TT | + |
| 17 | Igfbp2 (mouse) | BG791384 | 100% | 300 | synthetic | OVA | + |
| 18 | IGF1 | M29644 | 93% | 210 | synthetic | OVA | + |
| 19 | Interleukin 2 | X01586 | 63% | 399 | synthetic | TT | + |
| 20 | Interleukin 5 | X04688 | 70% | 336 | synthetic | TT | + |
| 21 | Leptin | U43653 | 83% | 438 | synthetic | TT | − |
| 22 | MBTPS2 (hamster) | AF019612 | — | 300 | synthetic | TT | − |
| 23 | MCIP1 (mouse) | Q9JHG6 | 100% | 591 | cDNA | TT | + |
| 24 | MCIP1 exon 1 | U28833 | 96% | 60 | synthetic | TT | + |
| 25 | MCIP1 exon 4 | U28833 | 96% | 60 | synthetic | OVA | − |
| 26 | MCIP2 exon 1 | U28833 | 96% | 60 | synthetic | OVA | + |
| 27 | MCIP2 exon 2 | U28833 | 96% | 60 | synthetic | OVA | + |
| 28 | MCIP3 exon 1 | U28833 | 96% | 60 | synthetic | OVA | + |
| 29 | MLCK1 | U48959 | 33% | 60 | synthetic | TT | + |
| 30 | MLCK4 | U48959 | 33% | 42 | synthetic | TT | + |
| 31 | R26W (mouse) | NM025960 | 100% | 300 | synthetic | TT | + |
| 32 | RYR2 | X98330 | 97% | 60 | synthetic | OVA | + |
| 33 | TBP | X54993 | 91% | 300 | synthetic | TT | + |
| 34 | TNFβ | QWHUX | 73% | 300 | synthetic | TT | + |
| 35 | TRPC2 (mouse) | NM011644 | 100% | 300 | synthetic | OVA | + |
| 36 | Ubiquitin (mouse) | NM018955 | 100% | 228 | cDNA | TT | + |
| 37 | VR1α | 2102273A | 84% | 51 | synthetic | OVA | − |

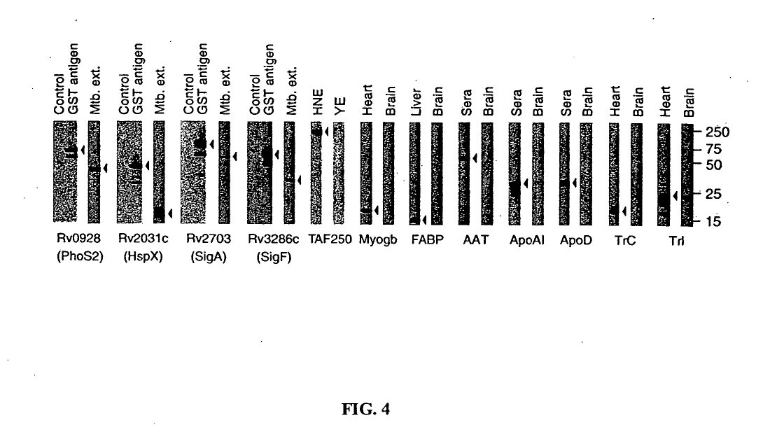

Protein was successfully overproduced in E. coli for 97% of the genes. Antibodies were produced after an average of two immunizations. Antigens identical to mouse sequences were as successful as antigens with lower identity, and there was no major difference in success rate between the two T cell epitope vectors. There are previous reports of producing antibodies against self-proteins by fusing T cell epitopes (King et al. 1998; Dalum et al., 1996), and the inventors have shown that this approach appears to work with many self-proteins. Four of the five antigens that previously failed to induce antibodies in pBQAP10 now produced antibodies. Furthermore, four antigens that previously failed to produce antibodies when delivered as protein now produced antibodies (ApoAV, R26W, RYR2, Ub). Overall 87% of large antigens (≧70 residues) and 79% of the small antigens (≦20 residues) produced antibodies, with an overall success rate of 84% (Table 2 and FIG. 4). There are few published studies with which the antibody production method developed in this study can be compared. The largest study to date is one that used protein immunizations with 570 antigens from Neisseria meningitidis (Pizza et al., 2000). Only 350 of the proteins could be overexpressed in E. coli and of those only 85 (24%) producing “strongly positive” antibodies. Another large study with a set of 40 synthetic peptides linked to keyhole limpet hemocyanin obtained a 63% success rate (Field et al., 1998).

To investigate possible causes of failure in our system the inventors tested sera for antibodies against the antigenic tag. Eight out of eight sera with antibodies against the test antigen also contained antibodies against the tag. Eight out of ten sera that did not contain antibodies against the test antigen did contain antibodies against the tag. Therefore, the inventors can eliminate many non-immunological causes of antibody response failure such as sub-optimal bullet preparation, plasmid delivery, protein translation, and protein secretion. Remaining possible causes of failure include post-translational modification of the antigen, structural features of the antigen, and B cell unresponsiveness. Sera were also tested for antibodies against other regions of the scaffold. The inventors did not detect antibodies to the COMP domain nor to the tetanus toxin epitopes, and only one out of seven samples had antibodies against the ovalbumin epitope (data not shown).

### Example 4

### Testing the Sensitivity of Antibodies Produced

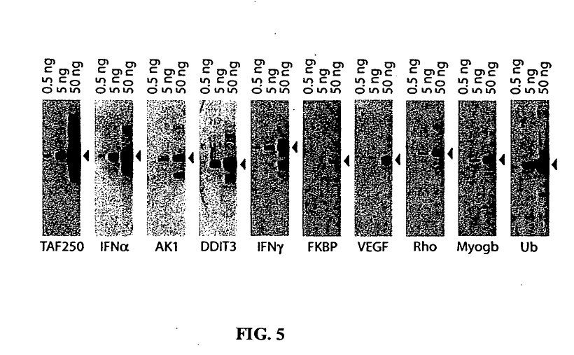

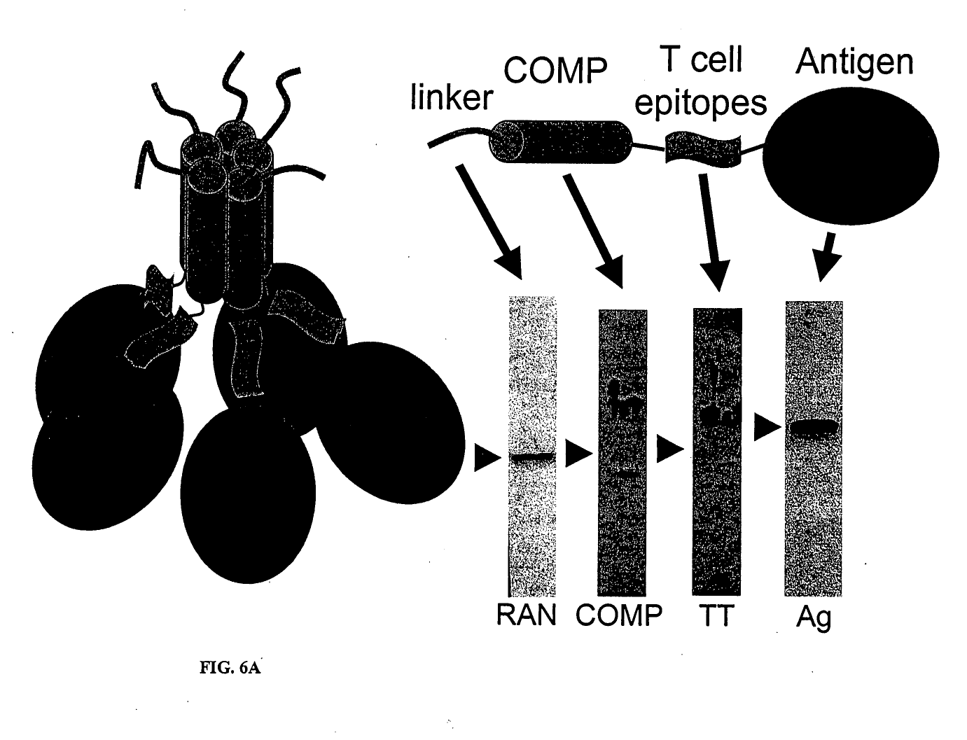

To examine whether the antibodies produced were useful for measuring the natural antigen, twelve of the antibodies were used to probe biological samples where the antigen was known to be expressed. All twelve antibodies detected a protein of the correct size in the appropriate sample, but not in a control sample (FIG. 4). Sensitivity was tested with randomly selected antibodies by titrating the corresponding GST fusion proteins on a western blot. Most of the antibodies could detect as little as a few nanograms of the GST-protein, including those raised against self-proteins (FIG. 5).

Although antibodies were obtained against up to 84% of the gene products that could be expressed in E. coli, a number of caveats should be mentioned. First, protein synthesis in at least one system is required to test these antibodies. While the proteins do not need to be purified, a great advantage over alternative methods, they do need to be made, as confirmation of specificity cannot be made without a protein source. If this is taken into account, the success rate is somewhat reduced to 82% for the small difficult antigens expressed with T cell epitopes, and 62% for the antigens expressed without the T cell epitope. Overall 90% of the 133 different antigens were successfully overexpressed in E. coli. This is a higher success rate than reported by other large-scale expression studies (Pizza et al., 2000; Braun et al., 2002). This higher success rate may largely be attributed to selecting small soluble fragments of proteins as well as avoiding membrane proteins or at least the membrane-associating region. Membrane proteins are typically the most difficult to overexpress, and it should be noted that half of the proteins that the inventors failed to express in E. coli were membrane proteins. Secondly, 21% of the sera (FIG. 2) showed some cross-reactivity with unexpected proteins in E. coli extracts supplemented with an irrelevent GST-fusion protein. There is no indication that these sera will react with antigens from the same organism as the one used for genetic immunization, however, this finding shows a relatively high rate of spurious cross-reaction, which should always be borne in mind when testing these, or indeed any polyclonal, sera.

High-throughput genomic technologies currently produce complete genome sequences and allow the measurement of entire mRNA populations. While these innovations have revolutionized biology, their impact will be limited unless the information generated can be translated to the protein level in a correspondingly high-throughput manner. The inventors have developed a high-throughput system for generating antibodies that can help close the gap. Application of this system could range from small scale analysis of interesting gene sets discovered by microarray analysis, to systematically generating antibodies against all putative proteins discovered in genome sequencing projects. Each CD1 mouse generates up to 2 mls of serum, sufficient for hundreds of immunoassays. Spleens from the mice can be saved so that larger amounts of highly valuable antibodies could later be generated as monoclonal or single chain antibodies (Barry et al., 1994; Chowdhury et al., 1998).

### Example 5

### Genetic Immunization Vectors Containing COMP

COMP is a pentameric glycoprotein of the thrombospondin family that is synthesized by cartilage and tendon. Its small oligomerizing domain is positioned at the N-terminus of the protein. Previous studies have shown that fusion of this domain to another protein can lead to chimeric pentamers inside the cell.

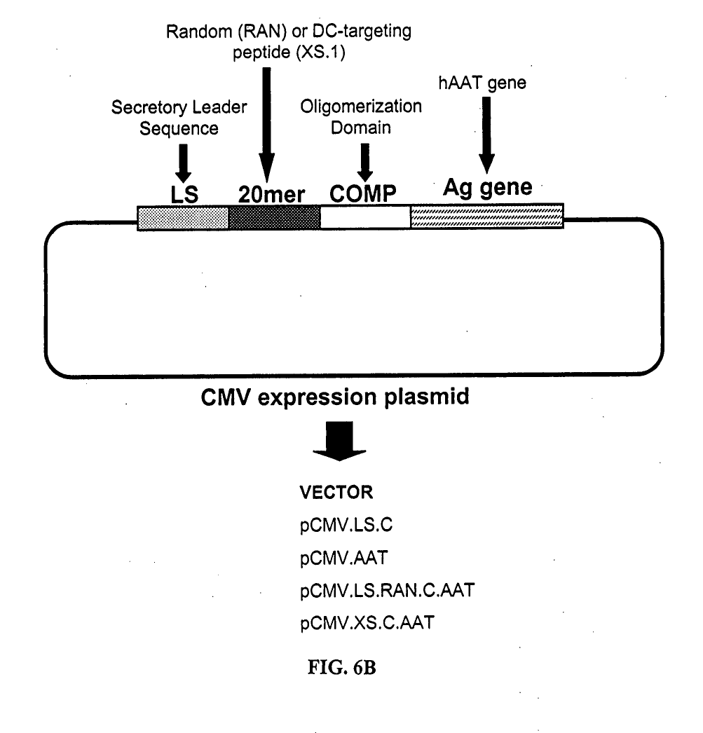

To determine whether COMP would be an effective adjuvant for antigens, plasmid vectors that can express inserted antigen genes as fusions with the short COMP pentamerization-domain were constructed. The genetic immunization vector, a CMV expression plasmid, contained the following sequences linked in cis, in a 5′ to 3′ direction: a secretory leader sequence (LS) from the human alpha-1-antitrypsin (hAAT) gene; a peptide sequence; the sequence of the cartilage oligomeric matrix protein domain (COMP) and an antigenic sequence (FIGS. 6A and 6B)

### Example 6

### Testing of COMP Genetic Immunization Vector

In order to test the ability of COMP to act as an adjuvant several different constructs were introduced into mice. These constructs were as follows: vector alone, contained the CMV expression plasmid with no LS, peptide, COMP or Ag gene; the pCMV.LS.C vector contained the CMV expression plasmid with, LS and COMP; the pCMV.AAT vector contained the AAT Ag alone; the pCMV.LS.RAN.C.AAT vector contained the CMV expression plasmid with, LS, RAN, COMP and the AAT Ag; and the pCMV.XS.C.AAT vector contained the CMV expression plasmid with the XS peptide, COMP, and the AAT Ag. The RAN peptide was used as a linker; it does not have any targeting function. The XS peptide specifically targets dendritic cells (DCs), which are key antigen-presenting cells.

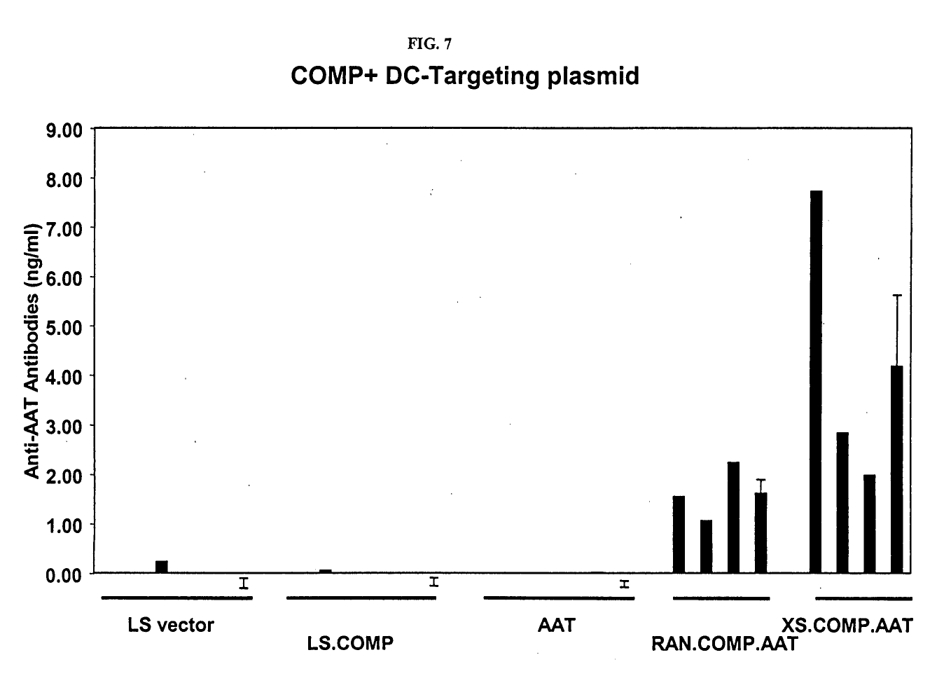

Five different groups of mice were genetically immunized with each of the CMV expression constructs as described in FIG. 6B (1 μg DNA per mouse) using the gene gun method and tested for alpha anti-trypsin (AAT) antibodies by ELISA (FIG. 7). Mice were bled 21 days post-immunization, and the specific anti-AAT levels are shown in the histogram.

The 3 control groups of animals (LS-vector alone, LS-COMP vector, and AAT-vector, corresponding to Group 1, 2 and 3 in FIG. 7) did not give rise to significant antibody levels. Group 4 mice containing the antigen (AAT) linked to COMP plus a non-targeting linker (RAN) gave rise to a measurable antibody levels. This indicates that COMP is important for giving rise to a specific immune response. Group 5 mice containing the antigen (AAT) liked to COMP plus a DC-targeting peptide (XS) gave rise to even higher antibody levels than those observed for Group 4, indicating that the XS targeting peptide can further increase the level of the specific immune response.

### Example 7

### COMP Increases Specific Antibody Levels

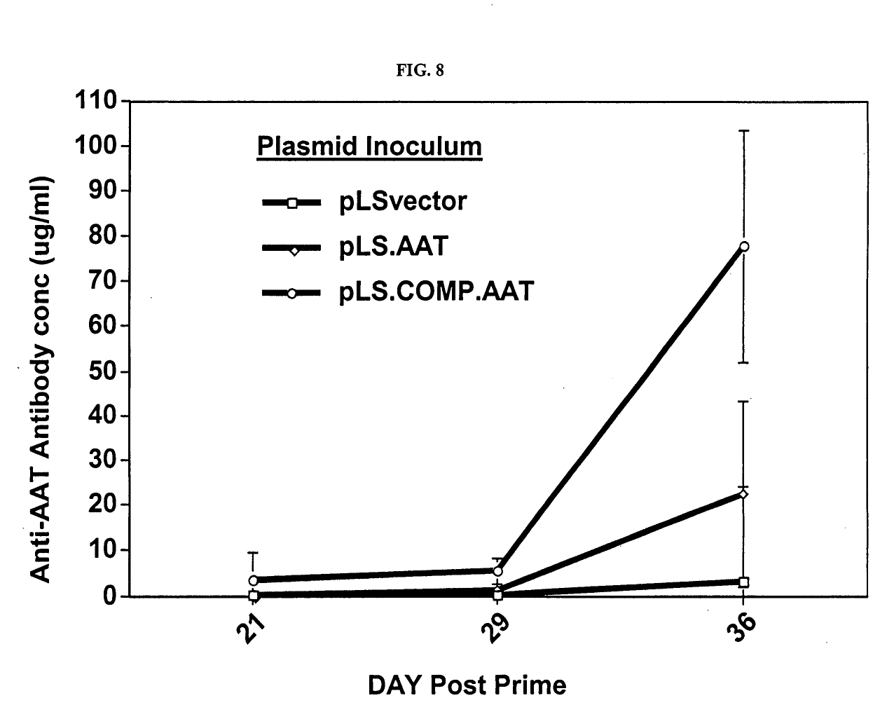

As shown in FIG. 8, groups of mice (5 per group) were immunized with the i) LS-vector control, ii) the vector containing the AAT antigen alone, and iii) the construct containing the AAT antigen plus COMP and the non-targeting RAN linker (Groups 1, 3 and 4, respectively). Mice were bled at 21, 29 and 36 days post-immunization, and the levels of specific anti-AAT antibodies are shown in the graph. The vector control (Group 1) did not give rise to significant antibody levels. The “AAT antigen alone” control (Group 3) gave rise to antibody levels of ˜20 μg/ml by day 36. The test group containing COMP, in addition the AAT and the RAN linker gave rise to ˜80μg/ml by day 36. Therefore the presence of COMP increased the specific antibody levels by ˜4 fold by day 36 post-immunization.

### Example 8

### Anti-AAT Antibody Levels Post-Immunization

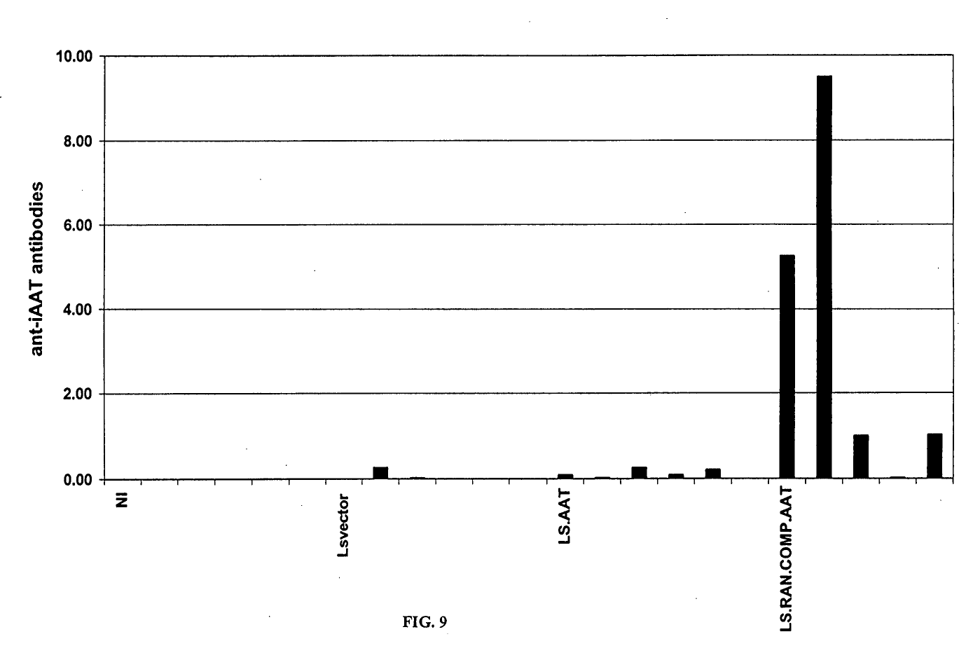

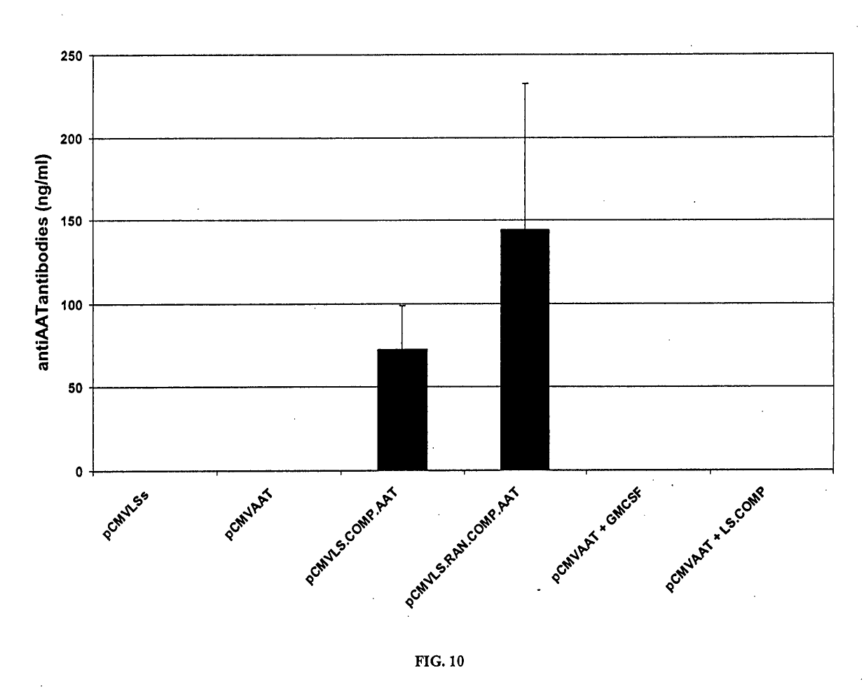

As shown in FIG. 9, groups of mice (5 per group) were immunized with the i) LS-vector control, ii) the vector containing the AAT antigen alone, and iii) the construct containing the AAT antigen plus COMP. In addition, one group was left unimmunized (NI). Measurement of anti-AAT antibody levels 6 weeks post-immunization showed that only the group that received that LS-RAN-COMP-AAT construct produced high titers.

### Example 9

### Generation of Significant Antibody Titers Using a COMP Linked in cis to a Antigen

As shown in FIG. 10, groups of mice (5 per group) were immunized with 1 μg of each of the following plasmids: i) LS-vector control, ii) the vector containing the AAT antigen alone, iii) AAT linked to COMP (ie. in cis) with a short 3 amino acid linker, iv) AAT linked to COMP and the RAN linker, v) the vector containing the AAT antigen alone co-delivered with the genetic adjuvant, GMCSF (SEQ ID NO:25), vi) the vector containing the AAT antigen alone co-delivered (ie. in trans) with LS-COMP. Anti-AAT antibody levels were measured 21 days post-immunization. The highest sera readouts of mice immunized with the LS-vector control were calculated as background levels. ELISAs were performed at 1:250 dilutions. The two groups that contained COMP linked in cis to the antigen showed significant antibody titers after 21 days. The addition of COMP in trans did not have this effect.

### Example 10

### COMP Causes an Elevated Humoral Response

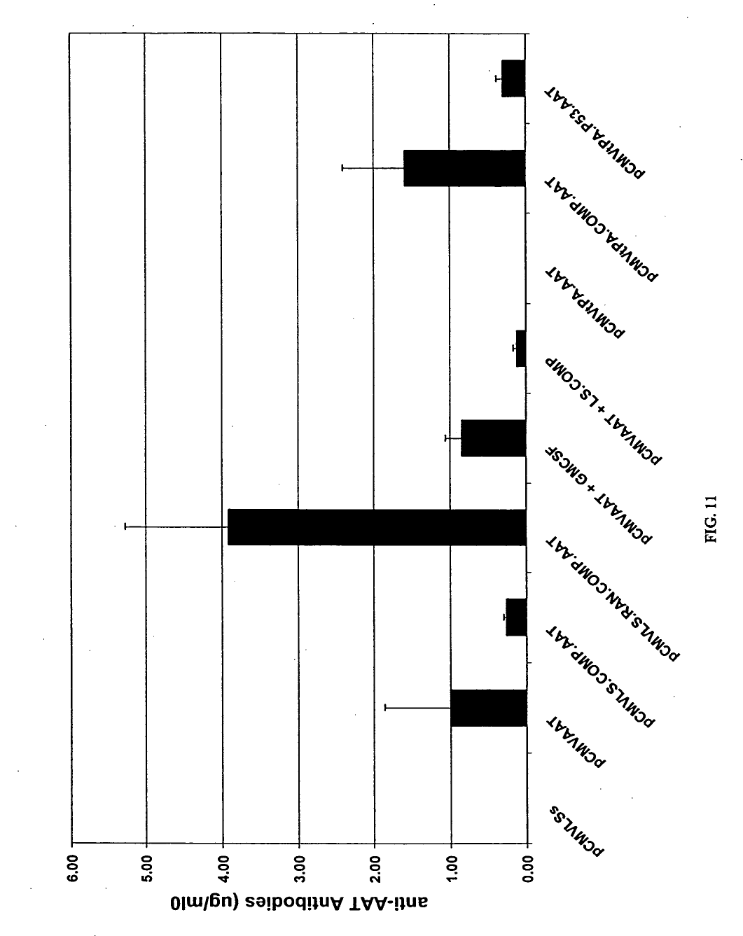

As demonstrated in FIG. 11, groups of mice (5 mice per group) were immunized with 1 μg of each of the following plasmids (left to right): i) the LS-vector, ii) the vector containing only the AAT antigen, iii) AAT linked in cis to COMP, iv) AAT linked in cis to COMP, joined by the RAN linker, v) the vector containing only the AAT antigen co-delivered with a plasmid encoding GMCSF, vi) the vector containing only the AAT antigen co-delivered with the LS-COMP vector, vii) a vector containing only the AAT antigen linked to the tPA leader sequence (in place of LS), viii) a vector containing the tPA-LS linked in cis to COMP and the AAT antigen, ix) a vector containing the tPA-LS linked in cis to the p53 oligomerization domain and the AAT antigen. Note that vectors viii and ix contain a 13 amino acid linker that is unrelated to RAN. Antibody titers were measured 28 days post-immunization.

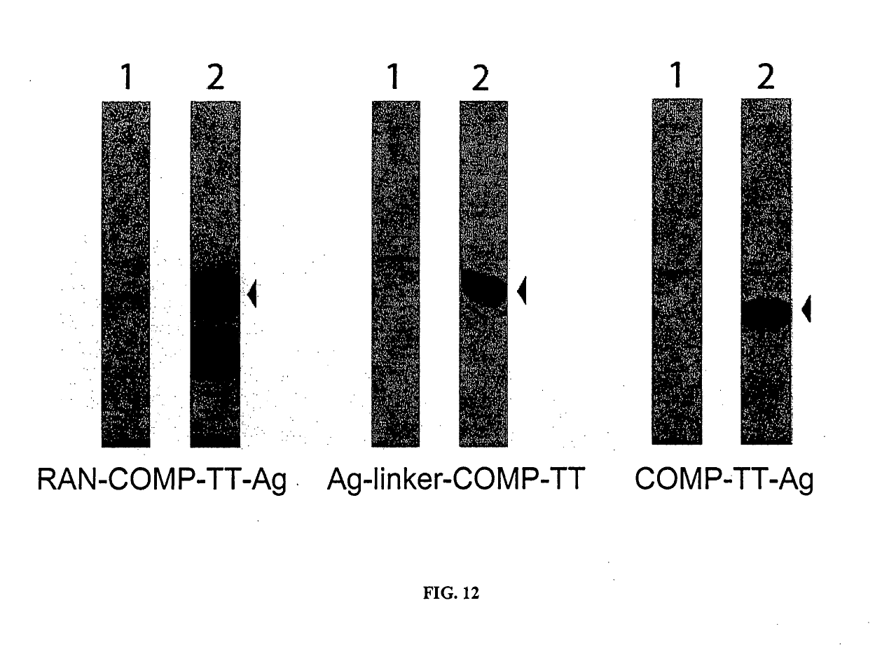

Significant antibody titers were observed with the following constructs: pCMV.COMP.AAT (indicating that COMP is important for an elevated humoral response); pCMV.RAN.COMP.AAT (indicating that the RAN linker is not required for the elevated humoral response at this early stage—compare with FIG. 12); pCMVtPA.COMP.AAT (indicating that the LS and tPA leader sequences are interchangeable); and pCMVtPA.p53.AAT (indicating that the p53 oligomerization domain is also effective in achieving elevated antibody levels)

### Example 11

### Measurement of Antibody Titers Following a Boost

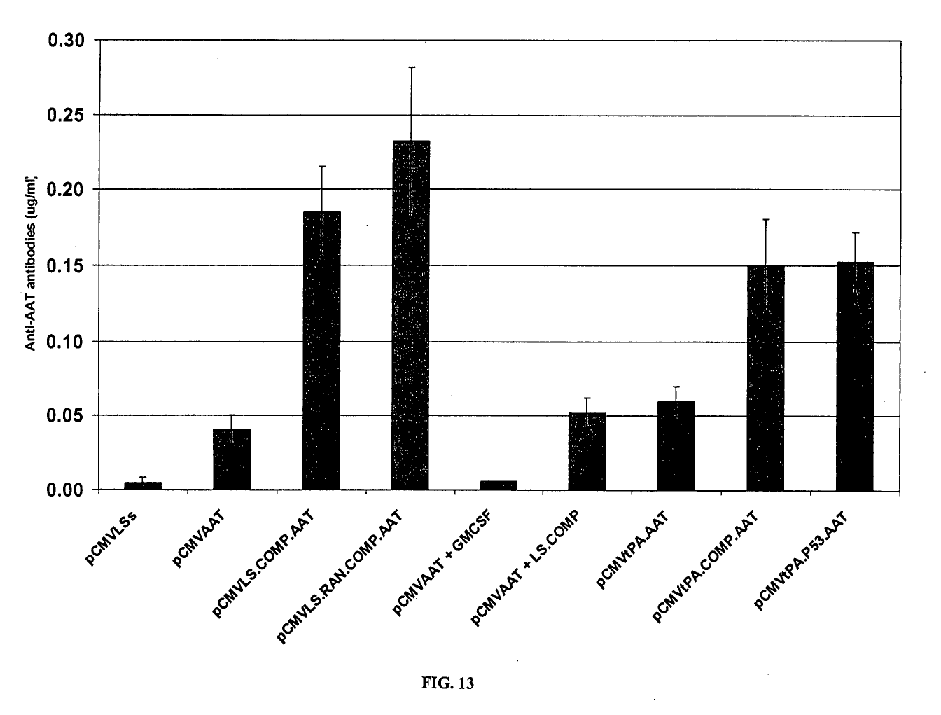

As described in above, groups of mice (5 per group) were immunized with 1 μg of each of the following plasmids (shown left to right): i) the LS-vector, ii) the vector containing the AAT antigen, iii) the vector containing AAT in cis with COMP, iv) the vector containing the RAN linker, COMP, and AAT, all linked in cis, v) the AAT vector co-delivered with the GM-CSG plasmid, vi) the AAT vector co-delivered with the LS-COMP plasmid (ie. in trans), vii) the tPA-AAT vector, viii) the tPA vector containing COMP and AAT in cis, ix) the tPA vector containing the p53 oligomerization domain linked in cis with AAT (FIG. 13).

This experiment was conducted in a similar manner to that described in FIG. 6, except in this case the antibody titers have been measured at a later time-point, following a boost. Sera were diluted 1:1000 for ELISA. In contrast to the results seen at the pre-boost earlier time-point, the presence of the RAN linker now seems to make a significant difference in enhancing the titer relative to the AAT and COMP.AAT groups.

### Example 12

### Antibody Production in Chickens

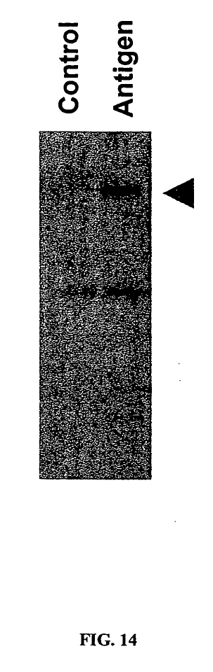

A genetic immunization plasmid containing a COMP-antigen fusion was immunized into a group of 2 chickens. Antibodies were isolated from egg yolks and used to probe the antigen on a western blot. The antibodies detected a species on the blot of the appropriate molecular size (arrow) but not in a control lane that did not contain the antigen (FIG. 14).

All of the compositions and methods disclosed and claimed herein can be made and executed without undue experimentation in light of the present disclosure. While the compositions and methods of this invention have been described in terms of preferred embodiments, it will be apparent to those of skill in the art that variations may be applied to the compositions and methods and in the steps or in the sequence of steps of the methods described herein without departing from the concept, spirit and scope of the invention. More specifically, it will be apparent that certain agents which are both chemically and physiologically related may be substituted for the agents described herein while the same or similar results would be achieved. All such similar substitutes and modifications apparent to those skilled in the art are deemed to be within the spirit, scope and concept of the invention as defined by the appended claims.

References

The following references, to the extent that they provide exemplary procedural or other details supplementary to those set forth herein, are specifically incorporated herein by reference.
U.S. Pat. No. 4,578,770
U.S. Pat. No. 3,791,932
U.S. Pat. No. 3,949,064
U.S. Pat. No. 4,174,384
U.S. Pat. No. 4,554,101
U.S. Pat. No. 4,596,792
U.S. Pat. No. 4,599,230
U.S. Pat. No. 4,599,231
U.S. Pat. No. 4,601,903
U.S. Pat. No. 4,608,251
U.S. Pat. No. 4,684,611
U.S. Pat. No. 4,952,500
U.S. Pat. No. 5,302,523
U.S. Pat. No. 5,322,783
U.S. Pat. No. 5,384,253
U.S. Pat. No. 5,399,363
U.S. Pat. No. 5,464,765
U.S. Pat. No. 5,466,468
U.S. Pat. No. 5,538,877
U.S. Pat. No. 5,538,880
U.S. Pat. No. 5,543,158
U.S. Pat. No. 5,550,318
U.S. Pat. No. 5,563,055
U.S. Pat. No. 5,580,859
U.S. Pat. No. 5,589,466
U.S. Pat. No. 5,610,042
U.S. Pat. No. 5,641,515
U.S. Pat. No. 5,656,610
U.S. Pat. No. 5,702,932
U.S. Pat. No. 5,705,629
U.S. Pat. No. 5,736,524
U.S. Pat. No. 5,780,448
U.S. Pat. No. 5,789,215
U.S. Pat. No. 5,846,225
U.S. Pat. No. 5,846,233
U.S. Pat. No. 5,945,100
U.S. Pat. No. 5,981,274
U.S. Pat. No. 5,989,553
U.S. Pat. No. 5,994,624
U.S. Pat. No. 6,410,241
U.S. patent appln. Ser. No. 10/077,508
U.S. patent appln. Ser. No. 10/077,392
U.S. patent appln. Ser. No. 10/077,247
U.S. patent appln. Ser. No. 10/077,232
U.S. patent appln. Ser. No. 10/077,621
U.S. Provisional Appl. Ser. No. 60/448,166
Andre et al., J. Virol., 72:1497-1503, 1998.
Ausubel et al., In: Current Protocols in Molecular Biology, John, Wiley & Sons, Inc, New York, 1994.
Babiuk et al., Vet. Immunol. Immunopathol., 72:189-202, 1999.
Barry et al., Biotechniques, 16:616-619, 1994.
Braun et al., Proc. Natl. Acad. Sci. USA, 99:2654-2659, 2002.
Chambers et al., Nat. Biotechnol., 21(9):1088-92, 2003.
Chang et al., J. Biol. Chem., 276:30956-30963, 2001.
Chen and Okayama, Mol. Cell Biol., 7(8):2745-2752, 1987.
Chowdhury et al., Proc. Natl. Acad. Sci. USA, 95:669-674, 1998.
Cohen et al, FASEB J, 12(15):1611-1626, 1998.
Coupar et al., Gene, 68:1-10, 1988.
Dalum et al., J. Immunol., 157:4796-4804, 1996.
European Appln. EP 266,032
Fechheimer et al., Proc. Natl. Acad. Sci. USA, 84:8463-8467, 1987.
Field et al., Methods Enzymol., 298:525-541, 1998.
Fraley et al., Proc. Natl. Acad. Sci. USA, 76:3348-3352, 1979.
Friedmann, Science, 244:1275-1281, 1989.
Froehler et al., Nucleic Acids Res., 14(13):5399-5407, 1986.
Fynan et al., Proc. Natl. Acad. Sci. USA, 90(24):11478-11482, 1993.
Gopal, Mol. Cell Biol., 5:1188-1190, 1985.
Graham and Van Der Eb, Virology, 52:456-467, 1973.
Hammond and Helenius, Curr. Opin. Cell Biol., 7:523-529, 1995.
Harlan and Weintraub, J. Cell Biol., 101:1094-1099, 1985.
Hermonat and Muzycska, Proc. Natl. Acad. Sci. USA, 81:6466-6470, 1984.
Horwich et al. J. Virol., 64:642-650, 1990.
International Human Genome Sequencing Consortium, Nature, 409:860-921, 2001.
Jameson and Wolf, Comput. Appl. Biosci., 4:181-186, 1988.
Johnson et al., In: Biotechnology And Pharmacy, Pezzuto et al. (Eds.), Chapman and Hall, NY, 1993.
Kaeppler et al., Plant Cell Reports, 9:415-418, 1990.
Kaneda et al., Science, 243:375-378, 1989.
Kato et al, J. Biol. Chem., 266:3361-3364, 1991.
King et al., Nat. Med., 4:1281-1286, 1998.
Kodadek, Chem. Biol., 8:105-115, 2001.
Kyte and Doolittle, J. Mol. Biol., 57(1):105-32, 1982.
Li et al., Infect. Immun., 67:4780-4786, 1999.
Maniatis, et al., Molecular Cloning, A Laboratory Manual, Cold Spring Harbor Press, Cold Spring Harbor, N.Y., 1990.
Nicolas and Rubinstein, In: Vectors: A survey of molecular cloning vectors and their uses, Rodriguez and Denhardt (Eds.), Stoneham: Butterworth, 494-513, 1988.
Nicolau and Sene, Biochim. Biophys. Acta, 721:185-190, 1982.
Nicolau et al., Methods Enzymol., 149:157-176, 1987.
Niwa et al., Gene, 108:193-199, 1991.
Omirulleh et al., Plant Mol. Biol., 21(3):415-28, 1993.
PCT Appln. WO 00/01801
PCT Appln. WO 94/09699
PCT Appln. WO 95/06128
PCT Appln. WO 98/18943
Pertmer et al., J. Virol., 70(9):6119-6125, 1996.
Pertmer et al., Vaccine, 13(15):1427-1430, 1995.
Pizza et al., Science, 287:1816-1820, 2000.
Potrykus et al., Mol. Gen. Genet., 199:183-188, 1985.
Potter et al., Proc. Natl. Acad. Sci. USA, 81:7161-7165, 1984.
Remington's Pharmaceutical Sciences, 15^{th} ed., pages 1035-1038 and 1570-1580, Mack Publishing Company, Easton, Pa., 1980.
Ridgeway, In: Vectors: A survey of molecular cloning vectors and their uses, Rodriguez and Denhardt (Eds.), Stoneham:Butterworth, 467-492, 1988.
Rippe et al., Mol. Cell Biol., 10:689-695, 1990.
Sambrook et al., In: Molecular cloning, Cold Spring Harbor Laboratory Press, Cold Spring Harbor, N.Y., 2001.
Scheerlinck, Vaccine, 19:2647-2656, 2001.
Sjolander et al., Mol. Immunol., 35(3):159-166, 1998.
Smith et al., PCR Methods Appl., 2:328-332, 1993.
St. Clair et al., Proc. Natl. Acad. Sci. USA, 96:9469-9474, 1999.
Stemmer et al., Gene, 164:49-53, 1995.
Stratford et al., Vaccine, 19:810-815, 2001.
Svanholm et al., J. Immunol. Methods, 228:121-130, 1999.
Sykes and Johnston, DNA Cell Biol., 18:521-531, 1999.
Sykes and Johnston, Nat. Biotechnol., 17:355-359, 1999.
Tang et al., Nature, 356:152-154, 1992.
Temin, In: Gene Transfer, Kucherlapati (Ed.), NY, Plenum Press, 149-188, 1986.
Terskikh et al., Proc. Natl. Acad. Sci. USA, 94:1663-1668, 1997.
Tur-Kaspa et al., Mol. Cell Biol., 6:716-718, 1986.
Valenzuela et al., Nature, 298:347-350, 1982.
Wong et al., Gene, 10:87-94, 1980.
Wu and Wu, Biochemistry, 27:887-892, 1988.
Wu and Wu, J. Biol. Chem., 262:4429-4432, 1987.
Yin et al., J. Biol. Resp. Modif., 8:190-205, 1989.
Zinkernagel, Nat. Immunol., 1:181-185, 2000.

1. A method of initiating or enhancing an immune response in a subject comprising administering to a subject a nucleic acid comprising a sequence encoding a COMP domain and a nucleic acid comprising a sequence encoding an antigen domain.

2. The method of claim 1, wherein the sequence encoding a COMP domain and the sequence encoding an antigen domain are comprised in the same nucleic acid.

3. The method of claim 2, wherein COMP domain is functionally linked to the antigen domain.

4. The method of claim 2, wherein the nucleic acid encodes a fusion protein comprising the COMP domain and the antigen domain.

5. The method of claim 2, wherein the nucleic acid encodes a separate COMP domain and a separate antigen domain.

6. The method of claim 5, wherein the COMP domain serves as an adjuvant to initiate or enhance an immune response to the antigen.

7. The method of claim 6, wherein the immune response is directed against a disease.

8. The method of claim 6, wherein the immune response protects against a disease.

9. The method of claim 7, wherein the disease is a pathogenic infection, a viral infection, or cancer/malignancy.

10. The method of claim 6, wherein an antibody against the antigen is produced in the subject.

11. The method of claim 2, wherein the nucleic acid is further defined as a vector.

12. The method of claim 11, wherein the vector contains the COMP domain encoding nucleic acid and the antigen encoding nucleic acid in cis.

13. The method of claim 11, wherein the vector contains the COMP domain encoding nucleic acid and the antigen encoding nucleic acid in trans.

14. The method of claim 2, wherein the nucleic acid comprises sequences encoding multiple COMP domains and/or multiple antigen domains.

15. The method of claim 15, wherein the nucleic acid comprises two or three COMP domains.

16. The method of claim 14, wherein the nucleic acid is further defined as comprising multiple identical COMP domains.

17. The method of claim 14, wherein the nucleic acid is further defined as comprising multiple different COMP domains.

18. The method of claim 2, wherein the nucleic acid is further defined as comprising a promoter, an enhancer, a targeting peptide encoding domain, and/or a secretory peptide encoding domain.

19. The method of claim 18, wherein the nucleic acid is further defined as comprising a chemically synthesized promoter.

20. The method of claim 18, wherein the nucleic acid comprises a secretory leader sequence linked to the nucleic acid sequence comprising the COMP domain by a non-immunogenic peptide sequence.

21. The method of claim 20, wherein the non-immunogenic peptide is a cell-targeting peptide.

22. The method of claim 21, wherein the cell-targeting peptide is a dendritic cell-targeting peptide.

23. The method of claim 1, wherein the COMP domain comprises all or part of SEQ ID NO:1; SEQ ID NO:3; SEQ ID NO:4; SEQ ID NO:6; SEQ ID NO:8; SEQ ID NO:9; SEQ ID NO:11; SEQ ID NO:13; SEQ ID NO:15; SEQ ID NO:18, SEQ ID NO:19, or a sequence encoding SEQ ID NO:2, SEQ ID NO:5, SEQ ID NO:7, SEQ ID NO:10, SEQ ID NO:12, SEQ ID NO:14, SEQ ID NO:16 or SEQ ID NO:18.

24. The method of claim 1, wherein the COMP domain is a mutated or modified COMP domain.

25. The method of claim 1, wherein the nucleic acid encoding a COMP domain encodes a less than full-length segment of a COMP protein.

26. The method of claim 1, wherein the nucleic acid encoding a COMP domain encodes as COMP domain comprising at least 5 contiguous amino acids of a nucleic acid sequence encoding any full-length COMP polypeptide.

27. The method of claim 26, wherein the nucleic acid encoding a COMP domain encodes as COMP domain comprising at least 15 contiguous nucleic acids of the amino acid sequence of any full-length COMP polypeptide.

28. The method of claim 1, wherein the administration comprises inhalation, gene gun, or injection.

29. The method of claim 1, wherein the subject is a mammal or a bird.

30. The method of claim 29, wherein the subject is a human, rat, mouse, cow, pig, horse, goat, or chicken.

31. The method of claim 1, wherein the subject is a bird and the method further comprises obtaining antibodies from an egg of the chicken.

32. A nucleic acid comprising a sequence encoding a COMP domain and a sequence encoding an antigen domain.

33. The nucleic acid of claim 32, wherein COMP domain is functionally linked to the antigen domain.

34. The nucleic acid of claim 32, further defined as encoding a fusion protein comprising the COMP domain and the antigen domain.

35. The nucleic acid of claim 32, further defined as encoding a separate COMP domain and a separate antigen domain.

36. The nucleic acid of claim 32, wherein the antigen domain can produce an immune response against a disease.

37. The nucleic acid of claim 36, wherein the immune response protects against a disease.

38. The nucleic acid of claim 37, wherein the disease is a pathogenic infection, a viral infection, or cancer/malignancy.

39. The nucleic acid of claim 32, further defined as a vector.

40. The nucleic acid of claim 39, wherein the vector contains the COMP domain encoding nucleic acid and the antigen encoding nucleic acid in cis.

41. The nucleic acid of claim 39, wherein the vector contains the COMP domain encoding nucleic acid and the antigen encoding nucleic acid in trans.

42. The nucleic acid of claim 32, further defined as comprising sequences encoding multiple COMP domains and/or multiple antigen domains.

43. The nucleic acid of claim 42, further defined as comprising two or three COMP domains.

44. The nucleic acid of claim 42, further defined as comprising multiple identical COMP domains.

45. The nucleic acid of claim 42, further defined as comprising multiple different COMP domains.

46. The nucleic acid of claim 32, further defined as comprising a promoter, an enhancer, a targeting peptide encoding domain, and/or a secretory peptide encoding domain.

47. The nucleic acid of claim 46, further defined as comprising a chemically synthesized promoter.

48. The nucleic acid of claim 46, further defined as comprising a secretory leader sequence linked to the nucleic acid sequence comprising the COMP domain by a non-immunogenic peptide sequence.

49. The nucleic acid of claim 48, wherein the non-immunogenic peptide is a cell-targeting peptide.

50. The nucleic acid of claim 49, wherein the cell-targeting peptide is a dendritic cell-targeting peptide.

51. The nucleic acid of claim 32, wherein the COMP domain comprises all or part of SEQ ID NO:1; SEQ ID NO:3; SEQ ID NO:4; SEQ ID NO:6; SEQ ID NO:8; SEQ ID NO:9; SEQ ID NO:11; SEQ ID NO: 13; SEQ ID NO:15; SEQ ID NO:18, SEQ ID NO:19, or a sequence encoding SEQ ID NO:2, SEQ ID NO:5, SEQ ID NO:7, SEQ ID NO:10, SEQ ID NO:12, SEQ ID NO:14, SEQ ID NO:16 or SEQ ID NO:18.

52. The nucleic acid of claim 32, wherein the COMP domain is a mutated or modified COMP domain.

53. The nucleic acid of claim 32, wherein COMP domain is a less than full-length segment of a COMP protein.

54. The nucleic acid of claim 32, wherein the COMP domain comprises at least 5 contiguous amino acids of a nucleic acid sequence encoding any full-length COMP polypeptide.

55. The nucleic acid of claim 54, wherein the COMP domain comprises at least 15 contiguous nucleic acids of the amino acid sequence of any full-length COMP polypeptide.
